(19) United States
(12) Patent Application Publication
(10) Pub. No.: US 2015/0139961 A1
(43) Pub. Date: May 21, 2015

(54) Reprogramming Blood Cells to Pluripotent and Multipotent Stem Cells

(71) Applicant: The Johns Hopkins University, Baltimore, MD (US)

(72) Inventor: Linzhao Cheng, Columbia, MD (US)

(21) Appl. No.: 14/295,623

(22) Filed: Jun. 4, 2014

Related U.S. Application Data
(63) Continuation of application No. 13/395,916, filed on May 30, 2012, now abandoned.
(60) Provisional application No. 61/242,717, filed on Sep. 15, 2009.
(60) Provisional application No. 61/376,620, filed on Aug. 24, 2010.

(51) Int. Cl. C12N 15/85; C12N 5/074; A61K 35/28; G01N 33/50

(52) U.S. Cl. CPC C12N 15/85; G01N 33/5073; C12N 5/0696; A61K 35/28; G01N 2500/10
USPC 424/93.7; 435/455; 435/29; 435/325; 435/320.1

(57) ABSTRACT

The present invention is based on the seminal discovery that cord blood (CB) and adult bone marrow (BM) CD34+ cells can be reprogrammed to early stem cells. The invention provides the reprogramming of CB and adult bone marrow (BM) CD34+ cells from subjects without any pre-treatment. Provided are methods for reprogramming blood cells of a subject. Also provided are methods of disease modeling and methods of generating subject-specific differentiated cells. In addition, the invention provides methods of identifying an agent that alters a function of subject-specific differentiated cells as well as isolated pluripotent or multipotent stem cells reprogrammed from blood cells.

### BACKGROUND OF THE INVENTION

1. Field of the Invention

The present invention relates generally to the field of stem cells and more specifically to reprogramming blood cells to pluripotent and multipotent stem cells.

2. Background Information

Recent derivation of human induced pluripotent stem (iPS) cells from patients' somatic cells has made it possible to generate patient- and disease-specific stem cell lines for developing novel cell therapies and disease modeling. These human iPS cells exhibit characteristics similar to human embryonic stem (hES) cells including unlimited expansion in culture. Using vectors to deliver multiple transgenes encoding transcription factors such as OCT4, SOX2, KLF4 and c-MYC, most published protocols were for reprogramming adherent cells such as fibroblasts and keratinocytes from skin and hair.

It is also highly desirable to reprogram blood cells that are easily accessible and less exposed to environmental mutagens. For example, umbilical cord blood (CB) cells that are collected and stored in multiple cell banks could be used as a source of either autologous or allogeneic but histo-compatible iPS cell lines. More critically, the ability to reprogram blood cells is essential if one wishes to generate iPS cells containing somatic mutations that are restricted to the blood cells and found in acquired hematological disorders in order to investigate their pathogenesis. A previous study demonstrated that differentiated mouse B cells could be reprogrammed to iPS cells, primarily by using transgenic (reprogramming-ready) mice harboring the four reprogramming transgenes that are conditionally active. More recently mouse iPS cell lines were also derived from bone marrow progenitor cells obtained from a mouse whose hematopoiesis was reconstituted from a single congenic hematopoietic stem cell, providing further evidence that mouse hematopoietic cells can be reprogrammed to pluripotency.

Derivation of iPS cells from postnatal human blood cells has not been reported until recently when it was reported that granulocyte colony-stimulating factor (G-CSF) mobilized peripheral blood (PB) CD34+ cells from a healthy person could be reprogrammed to iPS cells. It is unclear, however, whether daily G-CSF treatment could affect the reprogramming process or the properties of blood cell-derived iPS cells.

Thus, there remains a need for generating induced pluripotent stem cells efficiently from somatic cells, especially blood cells or umbilical cord blood cells.

### SUMMARY OF THE INVENTION

The present invention is based on the seminal discovery that cord blood (CB) and adult bone marrow (BM) CD34+ cells can be reprogrammed to early stem cells. The invention provides the reprogramming of CB and adult bone marrow (BM) CD34+ cells from subjects without any pre-treatment. Provided are methods for reprogramming blood cells of a subject. Also provided are methods of disease modeling and methods of generating subject-specific differentiated cells. In addition, the invention provides methods of identifying an agent that alters a function of subject-specific differentiated cells as well as isolated pluripotent or multipotent stem cells reprogrammed from blood cells.

In one embodiment, the invention relates to a method for generating a pluripotent or multipotent stem cell. The method includes introducing a non-viral vector containing at least one pluripotency factor into a blood cell in culture, thereby reprogramming the blood cell to a pluripotent or multipotent stem cell. In one aspect, the vector includes a plurality of pluripotency genes. In another aspect, the method of the invention further includes adding at least one corticosteroid to the culture. In another aspect, the at least one corticosteroid is selected from the group consisting of aldosterone, beclometasone, betamethasone, cortisone, deoxycorticosterone, dexamethasone, fludrocortisone, hydrocortisone, methylprednisolone, prednisolone, prednisone, and triamcinolone. In an additional aspect, the at least one corticosteroid is dexamethasone.

In another aspect, the method of the invention further includes adding at least one cytokine to the culture. In an additional aspect, the at least one cytokine is selected from the group consisting of sertoli cell factor (SCF), Fms-related tyrosine kinase 3 ligand (FLT3 or FL), thrombopoietin (TPO), erythropoietin (EPO), and interleukin 3 (IL-3). In another aspect, the blood cells are cord blood (CB) cells, adult bone marrow (BM) CD34+ cells, adult peripheral blood (PB) cells, adult peripheral blood (PB) CD34+ cells, adult peripheral blood (PB) CD34+CD45+ cells, or adult peripheral blood mononuclear cells (PBMCs). In another aspect, the subject is a mammal In an additional aspect, the subject is human. In another aspect, the pluripotent or multipotent stem cells include hematopoietic stem cells. In another aspect, the subject has myeloproliferative disorders (MPDs). In another aspect, the at least one pluripotency factor includes at least three factors selected from the group consisting of SOX2, SOX7, SOX17, OCT4, Nanog, LIN28, c-Myc, KLF4, ESRRB, EBF1, C/EBPa, C/EBPβ, NGN3, PDX and MAFA, or active fragments thereof In another aspect, the at least one factor includes OCT4, SOX2, KLF4, and c-MYC, or active fragments thereof In another aspect, the at least one factor includes OCT4, SOX2, KLF4, MYC, LIN28, and SV40 T antigen, or active fragments thereof.

In one aspect, the non-viral vector is an episomal vector. In another aspect, the method further includes introducing a second episomal vector into the blood cells. In an additional aspect, the second episomal vector expresses SV40 T antigen (Tg). In another aspect, the first episomal vector includes a plurality of pluripotency genes operatively linked to at least one regulatory sequence for expressing the factors. In an additional aspect, the episomal vector is a oriP/EBNA1 plasmid. In another additional aspect, the PBMCs are from a sickle cell anemia patient. In another aspect, the PBMCs include SCDB003 cells.

In another embodiment, the invention relates to a method of identifying an agent having a therapeutic effect on cells of a subject. The method includes contacting the pluripotent or multipotent stem cells generated from any of the methods described above with a test agent, and detecting a change in a function in presence of the test agent as compared to the function in absence of the test agent. In another embodiment, the invention relates to a method of generating differentiated cells. The method includes inducing differentiation of the pluripotent or multipotent stem cells produced from any of the methods described above, thereby obtaining a population of differentiated cells. In one aspect, the differentiated cells include blood cells, muscle cells, neuronal cells, connective tissues, cardiomyocyte cells, megakaryocyte cells, endothelial cells, hepatocytes, nephrogenic cells, adipogenic cells, osteoblast cells, osteoclastic cells, alveolar cells, cardiac cells, intestinal cells, renal cells, retinal cells or epithelial cells. In another aspect, the differentiated cells include pancreatic beta cells, hepatocytes, cardiomyocytes, or skeletal muscle cells.

In another embodiment, the invention relates to an enriched population of isolated pluripotent or multipotent stem cells produced by any of the methods described above. In one aspect, the isolated pluripotent or multipotent stem cells express a cell surface marker selected from the group consisting of: SSEA1, SSEA3, SSEA4, TRA-1-60, and TRA-1-81.

In another embodiment, the invention relates to a method of treating a disease requiring replacement or renewal of cells. The method includes administering to a subject an effective amount of the pluripotent or multipotent stem cells generated by any of the methods described above. In another embodiment, the invention relates to a reprogramming non-viral episomal vector. In one aspect, the non-viral episomal vector includes at least five pluripotency genes operatively linked to at least one regulatory sequence for expressing at least five pluripotency factors. In another aspect, the vector is a oriP/EBNA1 plasmid.

### BRIEF DESCRIPTION OF THE DRAWINGS

FIG. 1 shows gene expression of undifferentiated (OCT4 and NANOG) and differentiation markers in undifferentiated iPS cells (U) derived from CB and teratoma cells (T) after in vivo differentiation. The expression of alpha-fetoprotein (AFP, endoderm), CD34 (mesoderm) and PAX6 (ectoderm) and a housekeeping gene GAPDH can be measured by RT-PCR analysis.

FIGS. 2A-2D show increased erythroid differentiation of hematopoietic progenitor cells generated from PV iPS cells. To assess erythroid differentiation potential, purified CD34+CD45+ cells from both normal control (NC) and PV iPS cells after embryoid body-mediated hematopoietic differentiation are plated into a liquid culture medium (FIG. 2A and 2C), or a serum- and methylcellulose-containing medium (FIG. 2B and 2D). FIG. 2A shows folds of cell expansion after 7 days of the liquid culture from the purified CD34+CD45+ cells derived from NC (13.5±3.6 folds) or PV iPS cells (27.6±3.3 folds). FIG. 2B shows folds of cell expansion of the CD34+CD45+ cells after 14 days of the methylcellulose culture, 69.5±24.7 folds of NC vs. 127±28.3 folds of the PV iPS cells. Data in FIG. 2A and 2B are presented as mean±SD (n=2). FIG. 2C shows FACS analysis of the 7-day cultured cells for the erythroid phenotype (CD235a+CD45−). The percentages of such cell population are indicated in the upper left quadrant based on the gating and comparison with background staining. FIG. 2D shows FACS analysis of the 14-day cells harvested from the methylcellulose-containing medium.

FIGS. 3A-3B show human iPS generated hematopoietic progenitor cells exhibiting unique gene expression pattern similar to the primary CD34+ cells from the PV patient and a normal control. FIG. 3A shows total RNA isolated from primary CD34+ cells from a healthy donor as a normal control (NC) or from the PV patient (MPD183) where the PV-iPS cell lines are derived from. Gene expression of Nuclear Factor I-B (NFI-B), hemoglobin-gamma (HBG) and hemoglobin-beta (HBB) as well as beta-actin (as a control) can be analyzed by real-time quantitative PCR after reverse transcription of RNA. The normalized level (relative to that of beta-actin) is plotted. FIG. 3B shows an identical analysis of purified CD34+CD45+ cells generated from PV iPS (iMPD183) cells and a normal control (NC) iPS cells derived from normal adult CD34+ cells. Data are presented as mean±SD (n=2).

FIGS. 4A-4B show exemplary EBNA1/OriP episomal vectors for reprogramming human neonatal fibroblasts. FIG. 4A shows the configurations of vectors OSNL (Oct4+Sox2+Nanog+Lin28), OSTK (Oct4+Sox2+SV40Large T antigen+Klf4), and OSMK (Oct4+Sox2+c-Myc+Klf4). FIG. 4B shows a reprogramming example using the three plasmids.

FIG. 5 shows an exemplary illustration for reprogramming human blood CD34+ cells by improved EBNA1/oriP plasmids. Typically after day 14, the cells can be stained using an antibody against TRA-1-60+. Cells can then be tested for karyotype, pluripotency, and/or episome status.

FIGS. 6A-6B show that vector DNA diminishes in reprogrammed & expanded iPS cells. FIG. 6A suggests that the non-detection of the vector DNA can be due to epigenetic silencing of ENBA1 expression which is required for episomal DNA replication. FIG. 6B shows PCR results for the detection of the EBNA1 vectors.

FIG. 7 shows numbers of colonies from reprogramming CB MNCs, suggesting young blood can be better.

FIG. 8A shows genome-wide epigenetic signatures of 11 human ESC lines, 17 iPSC lines and their parental somatic cells. The InfiniumMethylation27 platform was used to analyze DNA methylation at 27,578 loci in IMR90 fetal fibroblasts and 3 adult bone marrow (BM) stromal cells (MSCs and BASC), 2 samples of isolated CD34+ cells from adult BM, peripheral blood (PB) and cord blood (CB). 17 iPSC lines derived from these somatic cells and 11 hESC samples were also used. FIG. 8A shows a dendrogram plot derived using Pearson correlation coefficients. It shows that iPSCs as a group are highly similar to ESCs and distinct to their parental somatic cells, and that human CD34+ cells are more similar to ESC/iPSC group as compared to the MSC/fetal fibroblasts.

FIG. 8B shows that K-means clustering analysis comes up a similar conclusion. To simplify presentation, the inventor omitted iPSCs since they are highly similar to ESCs. The level of promoter DNA methylation (from 0 to 1) of various loci (either within or outside a CpG island) of postnatal somatic cells and in 11 ESC lines is analyzed. Four distinct clusters are emerged: 1) high in both; 2) high in somatic cells but low in ESCs; 3) low in somatic cells but high in ESCs; and 4) low in both cell types. The numbers of loci in each cluster common in 3 MSCs or CD34+ cells are listed in the insert table. Clusters #2 and #3 consists of loci that are methylated differently from ESCs. Combining with the two clusters, 15.4% of loci in MSCs are different from ESCs and only 10.8% of loci in CD34+ cells are different from ESCs. FIG. 8C shows a multidimensional scaling analysis of the 2586 loci at ploycomb repressive complex target genes. A plot along the first two dimensions reveals that ESCs (and iPSCs, omitted from the plot) are indistinguishable from each other. Similarly, MSCs and CD34+ cells cluster separately. Notably, the CD34+ cell cluster is closer to ESCs in this aspect.

FIG. 8D shows reprogramming efficiency (measured as numbers of TRA-1-60+, ESC-like colonies at day 14 per 10⁶ nucleofected cells) by either Thomson/Yu combination (combo) #6 (3 plasmids expressing 7 genes) or the EBNA1/OriP combination, pEB-C5 (C5)+pEB-Tg (Tg). A pEB-GFP (GFP) was used as a control. FIG. 8E shows that the percentages of total colonies that are TRA-1-60+. Data are plotted as mean +/−SEM (n=2). FIGS. 8F and 8G show reprogramming efficiency of a different CD34+ cell sample by C5 with either Tg, pEB-p53shRNA (p53shRNA) or pEB-NANOG (NANOG), or C5 alone. Data are plotted as mean +/−SEM (n=3).

FIGS. 9A-9B show human iPSC derived from adult peripheral blood (PB) and bone marrow (BM) by a single ENBA-1/OriP plasmid. FIGS. 9A and 9B show number of TRA-1-60+ colonies per 10⁶ nucleofected cells expanded from PB CD34+ cells (FIG. 9A) or BM CD34+ cells (FIG. 9B). Data are plotted as mean+/−SEM, n=6.

FIGS. 10A-10B show human iPSC derived from adult peripheral blood (PB) and cord blood (CB) mononuclear cells (MNCs) by a single ENBA-1/OriP plasmid. FIG. 10A shows number of TRA-1-60+ colonies at day 14 after reprogramming of 2×10⁶ nucleofected cells expanded from CB MNCs. Two different CB MNCs, frozen 1.5 months ago (GB/CB) and 13.5 years ago (AC/CB) were cultured for 8 days. 2×10⁶ cultured and primed cells were nucleofected by 1-3 ENBA1/OriP plasmids as indicated. C5: pEB-C5; Tg: pEB-Tg; Combo #6: the Thomson/Yu combination #6 (3 plasmids). After additional culture for 2 days, the cells were plated onto 6 wells of 12-well plates coated with MEFs and cultured with ESC medium (starting at day 3) with or without sodium butyrate (NaB). After live staining for TRA-1-60 cell-surface expression at day 14, TRA-1-60+ (and TRA-60−) colonies were counted. Numbers of TRA-1-60+ colonies are plotted as mean+/−SEM, n=6. FIG. 10B shows PB MNCs (from donor SCD003) were similarly reprogrammed by the similar approach.

FIGS. 11A-11B shows phenotypes of expanded mononuclear cells (MNCs) from newborn cord blood (CB, A) or adult peripheral blood (PB, B). After 8 days or 9 days of culture in a defined serum-free culture to prime for reprogramming, cells are harvested for nucleofection by 1-2 plasmids and analyzed by FACS staining surface-marker staining. Phenotypes of MNCs before the culture (day 0) are also shown as controls. After culture (day 8 or 9), the majority of cells resemble erythroblasts that express a high level of CD71 (transferrin receptor), some of them also express intermediate levels of CD235a (glycophrin A). Very few cells express markers for T cells (CD3, <2.4%), B cells (CD19, <0/4%), monocytes (CD14, <0.4%) and granulocytes (CD15, <1.6%). Note that a cell population expressing a low-level of CD34 (2.5% to 6.8%) is also present.

FIG. 12 shows functions of expanded mononuclear cells (MNCs) from adult peripheral blood (PB). Hematopoietic progenitor assays for cells before (day 0) and after culture (day 8) are performed. After the day 8 culture, the frequencies of erythroid progenitors such as burst forming unit-erythrocytes (BFU-E, immature erythroid progenitors) and colony-forming unit-erythrocytes (CFU-E, more committed erythroid progenitors) increased greatly, while progenitors of colony-forming unit-granulocytes and monocytes) increased moderately. Colony-forming unit-momocytes and colony-forming unit-with mixed cell types (multipotent erythroid/myeloid progenitors) decreased.

FIGS. 13A-13B show fetal and adult hemoglobin expression patterns of expanded adult peripheral blood mononuclear cells (PBMCs). FIG. 13A shows RT-PCR to detect genes encoding hemoglobin gamma (HBB, fetal/newborn form) and hemoglobin beta (HBB, adult form) before (day 0) and after culture (day 9). Uncultured cord blood mononuclear cells (CBMCs) were used as a positive control for HBB and HBG mRNA levels. The mRNA from an iPSC line (SPE TNC1) reprogrammed from the same PBMCs (after the culture) was also included. After the culture for 9 days, the mRNA level of HBB increased >500-fold and HBG increased >8,000-fold. FIG. 13B shows FACS analysis of intracellular HBB and HBG proteins by specific monoclonal antibodies.

FIGS. 14A-14C show six SPE iPSCs lack any detectable somatic mutations associated with committed T cells and B cells. FIG. 14A shows that four sets of PCR primers were used to detect any evidence of T cell receptor (TCR) re-arrangement. Genomic DNA from a clonal human T cell line is provided as a positive control, while human ESC line (H1) serves as a negative control. Equal amounts of genomic DNA (500 ng) isolated from 6 iPSC lines derived from the PB MNCs (of donor SCD003) are used. None of them showed any positive signal of TCR re-arrangement. FIG. 14B shows that three sets of sets of PCR primers were used to detect any evidence of IgH re-arrangement occurring in committed B cells. A clonal control (29) was included as a positive control. None of six SPE iPSCs showed any positive signal of IgH re-arrangement, nor is the human ESCs. FIG. 14C shows quality controls of genomic DNA isolated from human ESCs and the six iPSCs. Mixed primers can readily amplify by PCR and generate multiple products from 100 by to 600 bp.

FIG. 15A shows that cultured blood CD34+ cells showing a signature closer to hES/iPS cells than that of fibroblasts/MSCs to hES/iPS cells. FIG. 15B shows that the adult PBMC-derived iPS cells are not from T cells with VDJ somatic mutations at the TCR locus. FIG. 15C shows a short summary for an exemplary embodiment of the invention where somatic/blood cells can be reprogrammed, expanded, and differentiated into various cell types.

### DETAILED DESCRIPTION OF THE INVENTION

Human induced pluripotent stem (iPS) cells derived from somatic cells hold promise to develop novel patient-specific cell therapies and research models for inherited and acquired diseases. Human adherent cells have been previously reprogrammed such as postnatal fibroblasts to iPS cells that resemble adherent embryonic stem cells. The invention provides derivation of iPS cells from postnatal human blood cells and the potential of these pluripotent cells for disease modeling. Multiple human iPS cell lines can be generated from previously frozen cord blood or adult CD34+ cells of healthy donors, and can be re-directed to hematopoietic differentiation. Multiple iPS cell lines can also be generated from peripheral blood CD34+ cells of two patients with myeloproliferative disorders (MPDs) who acquired the JAK2-V617F somatic mutation in their blood cells. The MPD-derived iPS cells containing the mutation appeared normal in phenotypes, karyotype and pluripotency. After directed hematopoietic differentiation, the MPD-iPS cell derived hematopoietic progenitor (CD34+CD45+) cells can show the increased erythropoiesis and gene expression of specific genes, recapitulating features of the primary CD34+ cells of the corresponding patient from whom the iPS cells are derived. These iPS cells provide a renewable cell source and a prospective hematopoiesis model for investigating MPD pathogenesis.

The invention provides the reprogramming of human CB and adult bone marrow (BM) CD34+ cells from healthy donors without any pre-treatment. Moreover, multiple iPS cell lines can be derived from PB CD34+ cells containing the JAK2-V617F mutation that is commonly found in hematopoietic progenitor cells of adult patients with myeloproliferative disorders (MPDs). The BCR/ABL-negative MPDs, which include polycythemia vera (PV), essential thrombocytosis (ET) and primary myelofibrosis (PMF), are a heterogeneous group of diseases characterized by increased proliferation of erythroid, megakaryocytic and myeloid lineages alone or in combination. The acquired common somatic mutation JAK2-V617F is present in >95% of PV, and ˜50% of ET and PMF patients. To determine if these blood cell-derived iPS cell lines can be used as a model to study normal and abnormal human hematopoiesis, an efficient serum-free differentiation protocol can be used to direct iPS cells into hematopoietic lineages. Similar to the increased erythropoiesis of hematopoietic progenitor (CD34+) cells isolated from PV patients, including one subject whose blood-derived iPS cells are used, re-differentiated hematopoietic progenitor (CD34+CD45+) cells generated from the PV-iPS cells can show enhanced erythropoiesis as compared to those from the iPS cells derived from normal CD34+ cells.

The term “corticosteroids,” as used herein, refers to a group of drugs similar to the natural corticosteroid hormones produced by the cortex of the adrenal glands. Corticosteroids are known to inhibit late phase allergic reactions via a variety of mechanisms. Corticosteroids include gluococorticoids and mineralocorticoids. In an aspect of the invention, a corticosteroid is selected from the group consisting of aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and pharmaceutically acceptable derivatives thereof.

In another aspect of the invention, a corticosteroid is selected from the group consisting of aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, and pharmaceutically acceptable derivatives thereof.

In one aspect of the invention, a cortocisteroid is a glucocorticoid selected from the group consisting of aldosterone, beclometasone, betamethasone, cortisone, deoxycorticosterone, dexamethasone, fludrocortisone, hydrocortisone, methylprednisolone, prednisolone, prednisone, and triamcinolone. In another aspect of the invention, a corticosteroid is dexamethasone.

The term “reprogramming,” as used herein, refers to a process where cells of a differentiated state are converted into cells of a de-differentiated state. Reprogrammed cells can be pluripotent or multipotent cells.

The term “pluripotent cells,” as used herein, refers to cells of a de-differentiated or undifferentiated state and can differentiate into various cell types. Pluripotent cells express pluripotent cell-specific markers, and have a cell morphology characteristic of undifferentiated cells (for example, compact colony, high nucleus to cytoplasm ratio, and/or prominent nucleolus). Typically pluripotent cells can be induced to differentiate into all three germ layers (e.g., endoderm, mesoderm and ectoderm). Teratoma formation can be observed in an immunocompromised animal, for example a SCID mouse, when pluripotent cells are introduced in vivo by a subcutaneous, intramuscular, or intratesticular route. (Thomson et al. (1998) Science 282:1145-47).

The term “pluripotency gene,” as used herein, refers to a gene that is associated with pluripotency of a cell. Typically a pluripotency gene is expressed only in pluripotent stem cells, and is crucial for the functional identity of pluripotent stem cells. The pluripotent stem cells are cells in a relatively undifferentiated state and can differentiate into different types of cells. The transcription factor OCT4 is an example of a pluripotency gene required for establishing and maintaining the undifferentiated phenotype of embryonic stem cells (Nichols et al. (1998) Cell 95:379-91; Niwa et al. (2000) Nature Genet. 24:372-76). Another exemplary pluripotency genes is Nanog (Chambers et al. (2003) Cell 113:643-55; Mitsui et al. (2003) Cell 113(5):631-42; Bortvin et al. (2003) Development 130(8):1673-80; Saitou et al. (2002) Nature 418 (6895):293-300). The term “pluripotency factor,” as used herein, refers to a factor expressed from a pluripotency gene.

The term “pluripotency characteristics” as used herein, refers to many characteristics associated with pluripotency, including, for example, the ability to differentiate into all types of cells and an expression pattern distinct for a pluripotent cell, including expression of pluripotency genes, expression of stem cell surface markers, and expression of biomarkers associated with pluripotency. For example, one reprogrammed cell can express alkaline phosphatase, but not express SSEA1, can proliferate for more than 30 passages, and can differentiating into hepatocytes or hematopoietic cells. Another reprogrammed cell, for example, can express SSEA1 on the cell surface, can be cultured for over a year without differentiation, and can also grow into endoderm, mesoderm, and ectoderm tissues.

Reprogrammed cells can be defined by presence of one or more surface markers or biomarkers. For example, some reprogrammed cells express alkaline phosphatase. Some reprogrammed cells express SSEA1, SSEA3, SSEA4, TRA-1-60, and/or TRA-1-81. Some reprogrammed cells express OCT4, SOX2, and Nanog. Expression of the surface marker or biomarkers can be determined at mRNA level or at protein level.

For another example, a reprogrammed cell can be positive for alkaline phosphatase and SSEA1 positive, but negative for SSEA4. Another reprogrammed cell can be positive for Nanog, SOX2, and OCT4. In an aspect of the invention, a reprogrammed cell can express cell surface antigens interacting with antibodies having the binding specificity to TRA-1-60 (for example, ATCC HB-4783) and/or TRA-1-81 (for example, ATCC HB-4784). Further, in another aspect of the invention, a reprogrammed cell can be maintained without a feeder layer.

A reprogrammed cell of the invention may have the potential to differentiate into a wide variety of cell types of different lineages including fibroblasts, osteoblasts, chondrocytes, adipocytes, skeletal muscle, endothelium, stroma, smooth muscle, cardiac muscle, neural cells, hemiopoetic cells, pancreatic islet, or other cell type. A reprogrammed cell of the invention may have the potential to differentiate into all cell lineages. A reprogrammed cell of the invention can differentiate into a number of lineages including 1, 2, 3, 4, 5, 6-10, 11-20, and greater than 20 lineages.

A pluripotency gene or pluripotency factor used by the methods of the invention includes but not limited to glycine N-methyltransferase (GNMT), Octamer-4 (OCT4), Nanog, GABRB3, LEFTB, NR6A1, PODXL, PTEN, SRY (sex determining region Y)-box 2 (also known as SOX2), Myc, REX-1 (also known as ZFP42), Integrin a6, ROX1, LIF-R, TDGF1 (CRIPTO), SALL4 (sal-like 4), Leukocyte cell derived chemotaxin 1 (LECTI), BUBI, FOXD3, NR5A2, TERT, LIFR, SFRP2, TFCP2L1, LIN28, XIST, and Kruppel-like factors (KLF) such as KLF4 and KLF5.

The methods of the invention can use 1, 2, 3, 4, 5, 6, 7, 8, 9, 10, 11-20, and greater than 20 pluripotency genes or pluripotency factors. In one aspect, the methods of the invention use at least three pluripotency genes or pluripotency factors. In another aspect, the methods of the invention use at least four pluripotency genes or pluripotency factors. In another aspect, the methods of the invention use at least five pluripotency genes or pluripotency factors. In another aspect, the methods of the invention use at least six pluripotency genes or pluripotency factors.

A test agent according to the methods of the invention can be, for example, a polynucleotide, a peptide, a peptidomimetic, peptoids such as vinylogous peptoids, a small organic molecule, or the like, and can act in any of various ways to alter a function of a cell. For example, the test agent can act extracellularly by binding to a cell surface receptor, thereby altering a function mediated by binding of a ligand that generally binds to and acts via the receptor. Alternatively, the test agent can be one that traverses cell membrane, either passively or via an active transport mechanism, and acts within a cell to alter a function.

A peptide test agent according to the methods of the invention can include from about two to four residues to hundreds or thousands amino acids. The term “peptide,” as used herein, does not suggest a particular size or number of amino acids comprising the molecule, and that a peptide test agent can contain up to several amino acid residues or more. Peptide test agents can be prepared, for example, by a method of chemical synthesis, or using methods of protein purification, followed by proteolysis and, if desired, further purification by chromatographic or electrophoretic methods, or can be expressed from an encoding polynucleotide. Further, a peptide test agent can be based on a known peptide, for example, a naturally occurring peptide, but can vary from the naturally occurring sequence, for example, by containing one or more D-amino acids in place of a corresponding L-amino acid; or by containing one or more amino acid analogs, for example, an amino acid that has been derivatized or modified at its reactive side chain.

Similarly, one or more peptide bonds in the peptide test agent can be modified, or a reactive group at the amino terminus or the carboxy terminus or both can be modified. Such peptides can have improved stability to a protease, an oxidizing agent or other reactive material the peptide test agent may encounter in a biological environment. Such peptide test agents also can be modified to have decreased stability in a biological environment where the period of time the peptide is active in the environment is reduced.

A polynucleotide test agent according to the methods of the invention can include from about two to four residues to hundreds or thousands nucleotides. The term “polynucleotide,” as used herein, is not limited to a sequence of two or more deoxyribonucleotides or ribonucleotides that are linked together by a phosphodiester bond. Linkages other than a phosphodiester bond can also be used.

The term “polynucleotide,” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide,” as used herein, includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond.

In general, the nucleotides comprising a polynucleotide are naturally occurring deoxyribonucleotides, such as adenine, cytosine, guanine or thymine linked to 2′-deoxyribose, or ribonucleotides such as adenine, cytosin, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al. (1994) Nucl. Acids Res. 22:5220-34; Jellinek et al. (1995) Biochemistry 34:11363-72; Pagratis et al. (1997) Nature Biotechnol. 15:68-73, each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al. (1994) Nucl. Acids Res. 22:977-86; Ecker and Crooke (1995) BioTechnology 13:351-60, each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al. (1995) Biochemistry 34:11363-72).

A polynucleotide test agent can be contacted with or introduced into a cell using methods as disclosed herein or otherwise known in the art. Generally, but not necessarily, the polynucleotide is introduced into the cell, where it effects its function either directly, or following transcription or translation or both. For example, the polynucleotide can encode a peptide test agent, which is expressed in a cell and alters a function of the cell. A polynucleotide test agent also can be, or can encode, an antisense molecule, an interfering RNA, a micro RNA, a ribozyme or a triplexing agent, which can be designed to target one or more specific target nucleic acid molecules.

Antisense polynucleotides, ribozymes and triplexing agents generally are designed to be complementary to a target sequence, which can be a DNA or RNA sequence, for example, mRNA, and can be a coding sequence, a nucleotide sequence comprising an intron-exon junction, a regulatory sequence such as a Shine-Delgarno sequence, or the like. The degree of complementarity is such that the polynucleotide, for example, an antisense polynucleotide, can interact specifically with the target sequence in a cell. Depending on the total length of the antisense or other polynucleotide, one or a few mismatches with respect to the target sequence can be tolerated without losing the specificity of the polynucleotide for its target sequence. Thus, few if any mismatches would be tolerated in an antisense molecule consisting, for example, of 20 nucleotides, whereas several mismatches will not affect the hybridization efficiency of an antisense molecule that is complementary, for example, to the full length of a target mRNA encoding a cellular polypeptide. The number of mismatches that can be tolerated can be estimated, for example, using well known formulas for determining hybridization kinetics (see Sambrook, J.; Fritsch, E. F. and Maniatis, T. “Molecular Cloning: A Laboratory Manual,” vol. I. 2^{nd} edition. Cold Spring Harbor Laboratory Press, 1989) or can be determined empirically using methods as disclosed herein or otherwise known in the art, particularly by determining that the presence of the antisense polynucleotide, ribozyme, or triplexing agent in a cell decreases the level of the target sequence or the expression of a polypeptide encoded by the target sequence in the cell.

A polynucleotide useful as an antisense molecule, a ribozyme or a triplexing agent can inhibit translation or cleave the nucleic acid molecule, thereby altering a function of a cell. An antisense molecule, for example, can bind to an mRNA to form a double stranded molecule that cannot be translated in a cell. Antisense oligonucleotides of at least about 15 to 25 nucleotides are preferred since they are easily synthesized and can hybridize specifically with a target sequence, although longer antisense molecules can be expressed from a polynucleotide introduced into the target cell. Specific nucleotide sequences useful as antisense molecules can be identified using well known methods, for example, gene walking methods (see, for example, Seimiya et al. (1997) J. Biol. Chez. 272:4631-36, which is incorporated herein by reference). Where the antisense molecule is contacted directly with a target cell, it can be operatively associated with a chemically reactive group such as iron-linked EDTA, which cleaves a target RNA at the site of hybridization. A triplexing agent, in comparison, can stall transcription (Maher et al. (1991) Antisense Res. Devel. 1:227; Helene (1991) Anticancer Drug Design 6:569).

A screening assay of the invention can be performed by contacting the test agent and cells in vivo, for example, following administration or implantation of the cells into a subject, or by contacting the test agent and cells in vitro, for example, by adding the test agent to a culture containing the cells or to cells isolated from a culture. The function of undifferentiated pluripotent stem cells that can be altered due to contact with an agent can be any function of the cells. For example, the function can be expression of gene that typically is expressed (or not expressed) in the cells, and the agent can alter the function by increasing or decreasing the level of expression of an expressed gene (e.g., decreasing expression of stage-specific surface antigen-4, alkaline phosphatase, or OCT4 transcription factor), or by turning on the expression of an unexpressed gene (e.g., inducing expression of stage-specific surface antigen-1), in the cells.

In one embodiment, the agent that effects a function of cells is one that induces differentiation of the cells, thereby producing differentiated cells. Such differentiated cells can be multipotent human stem cells (e.g., hematopoietic stem cells) or can be terminally differentiated cells (e.g., muscle cells, neuronal cells, blood cells, connective tissue, or epithelial cells). As such, the method of the invention can be used to identify an agent that induces differentiation of cells to pancreatic beta cells, hepatocytes, cardiomyocytes, skeletal muscle cells, or other cell types.

A screening method of the invention provides the advantage that it can be adapted to high throughput analysis and, therefore, can be used to screen combinatorial libraries of test agents in order to identify those agents that can alter a function of a pluripotent or multipotent cell. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith (1992) Science 249:386-90; Markland et al. (1991) Gene 109:13-19; each of which is incorporated herein by reference); a peptide library (U.S. Pat. No. 5,264, 563, which is incorporated herein by reference); a peptidomimetic library (Blondelle et al. (1995) Trends Anal. Chem. 14:83-92; a nucleic acid library (O'Connell et al. (1996) Proc. Natl. Acad. Sci., USA 93:5883-87; Tuerk and Gold (1990) Science 249:505-10; Gold et al. (1995) Ann. Rev. Biochem. 64:763-97; each of which is incorporated herein by reference); an oligosaccharide library (York et al. (1996) Carb. Res. 285:99-128; Liang et al. (1996) Science 274:1520-22; Ding et al. (1995) Adv. Expt. Med. Biol. 376:261-69; each of which is incorporated herein by reference); a lipoprotein library (de Kruif et al. (1996) FEBS Lett. 399:232-36, which is incorporated herein by reference); a glycoprotein or glycolipid library (Karaoglu et al. (1995) J Cell Biol. 130:567-77, which is incorporated herein by reference); or a chemical library containing, for example, drugs or other pharmaceutical agents (Gordon et al. (1994) J. Med. Chem. 37:1385-1401; Ecker and Crooke (1995) BioTechnology 13:351-60; each of which is incorporated herein by reference).

Polynucleotides can be particularly useful as agents that can alter a function of cells because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

For a high throughput format, cells of the invention can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the cells of the invention, particularly with respect to the function being examined An advantage of using a high throughput format is that a number of test agents can be examined in parallel, and, if desired, control reactions also can be run under identical conditions as the test conditions. As such, the methods of the invention provide a means to screen one, a few, or a large number of test agents in order to identify an agent that can alter a function of cells, for example, an agent that induces the pluripotent or multipotent cells to differentiate into a desired cell type, or that prevents spontaneous differentiation, for example, by maintaining a high level of expression of regulatory molecules such as OCT4.

The episomal vectors of the invention include components allowing the vector to self-replicate in cells. For example, the known Epstein Barr oriP/Nuclear Antigen-1 (EBNA-1) combination can support vector self-replication in mammalian cells, particularly primate cells. (Lindner and Sugden (2007) Plasmid 58(1):1-12, which is incorporated by reference in its entirety). Standard molecular biology techniques suitable for use in the subject invention for the construction of expression vectors are known to one of ordinary skill in the art and can be found in Sambrook et al., “Molecular cloning: a laboratory manual,” (3rd ed. Cold Spring harbor Press, Cold Spring Harbor, N.Y. 2001), which is incorporated by reference in its entirety.

A naturally occurring fatty acid, and house-hold nutritional supplement greatly enhance the efficiency of induced pluripotent stem (iPS) cell derived from human somatic cells. Novel bioactive molecules for reprogramming and treating the biological consequences of the underlying mutations are provided.

Provided are procedures to reprogram blood cells (from CB or adult PB) to pluripotent or multipotent stem cells, generating iPS cells that preserve the native and unaltered genome (without vector-insertion or somatic mutations).

Provided are methods to prepare (prime) PB and CB mononuclear cells for efficient reprogramming. Provided are a cocktail of cytokines and a hormone. In some embodiments, the inclusion of dexamethasone, a synthetic glucocorticoid, is a key to the success for efficient reprogramming. Provided are the use of dexamethasone and other glucocorticoids (native or synthetic) for reprogram somatic cells including blood cells, and for generation of iPS cells. Dexamethasone and other glucocorticoids have been used for establishing and expanding marrow stromal cells/ mesenchymal stem cells (multipotent stem cells or MSCs), and erythorid progenitor cells, but their use for reprogramming remains unknown until the subject invention.

Provided are the generations of iPS cell lines from CB, and adult blood and marrow CD34+ cells as well as their directed hematopoietic differentiation. The technology to derive iPS cells from cord blood provides the opportunity to generate histo-compatible stem cells for many individuals because of the large collections in cord blood banks. As in vitro expansion of hematopoietic stem/progenitor cells from CB and adult sources remains a challenge, unlimited expansion of derived iPS cells in combination with further optimized hematopoietic differentiation methods can provide a vital alternative way to store and amplify histo-compatible blood stem cells for blood/BM transplantation purposes. Reprogramming efficiency and properties of CD34+ cell-derived iPS cells with those derived from other postnatal cell types can be compared according to the invention.

The invention provides that MPD-specific iPS cell lines possessing somatic mutations that occur in blood cell lineages can be generated by the described reprogramming methods. The iPS cell lines from MPD (PV and PMF) patient peripheral blood can display the typical morphology and growth pattern as hES and iPS cells when being maintained as undifferentiated pluripotent stem cells. Upon induction, they can differentiate into various cell types originated from the three embryonic germ layers including hematopoietic cells. Notably, the re-differentiated hematopoietic progenitor cells from PV iPS cells can show similar increased erythropoiesis characteristic of the primary CD34+ cells from patients with PV, a hematopoietic disease signified by overproduction of red blood cells. A full characterization of two iPS cell lines derived from a PMF patient (MPD562) can also be provided according to the invention. Together, these iPS cell lines can provide a potential model system to study MPD pathogenesis.

Although the identification of JAK2-V617F mutation significantly advanced the understanding of MPD pathogenesis, questions still remain including how the JAK2-V617F clonal dominance occurs and how one mutation contributes to three different diseases. Transgenic mouse models suggest that a dosage effect of JAK2-V617F may contribute to different MPD phenotypes (Tiedt et al. (2008) Blood 111:3931-40; Xing et al. (2008) Blood 111:5109-17). Consistent with many previous studies indicating that other genetic or epigenetic events could be important to MPD development, recent studies demonstrate that a germ-line SNP is associated with predisposition to the development of JAK2-V617F+ MPDs. (Olcaydu et al. (2009) Nat Genet. 41:450-54; Kilpivaara et al. (2009) Nat Genet. 41:455-59; Jones et al. (2009) Nat Genet. 41:446-49). As a complement to these previous studies, the renewable iPS cell lines and subsequent hematopoietic differentiation technologies may provide a novel and prospective model to study MPD pathogenesis.

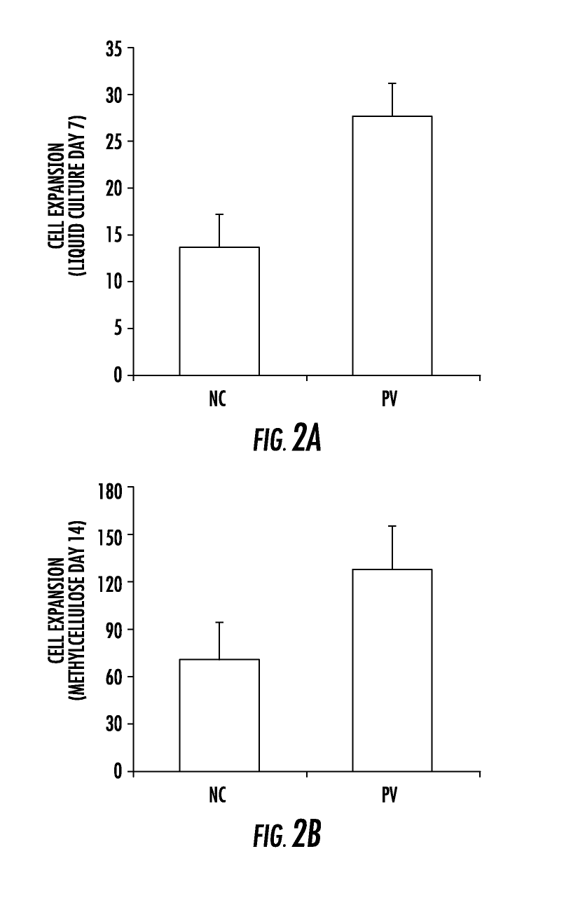

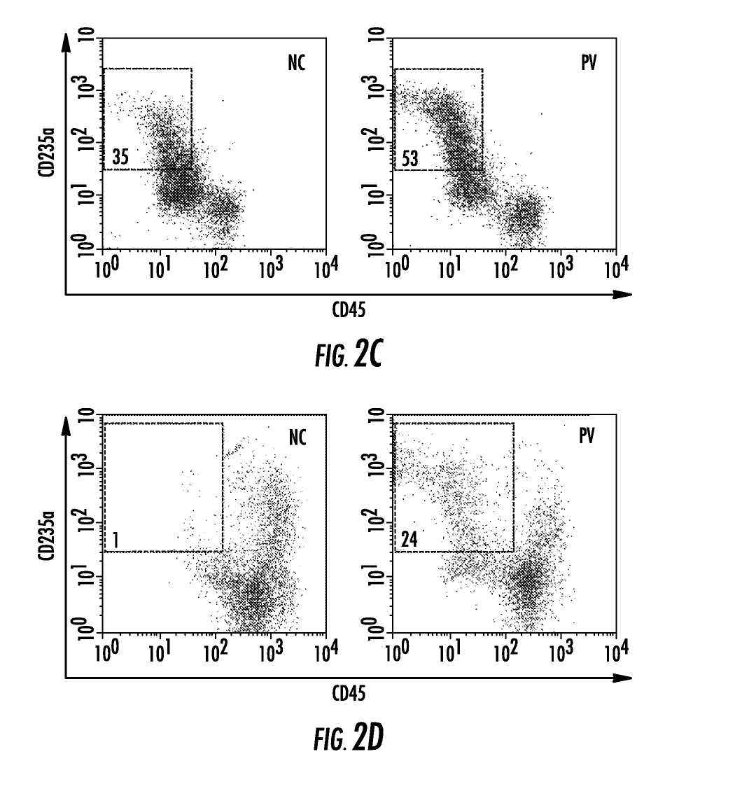

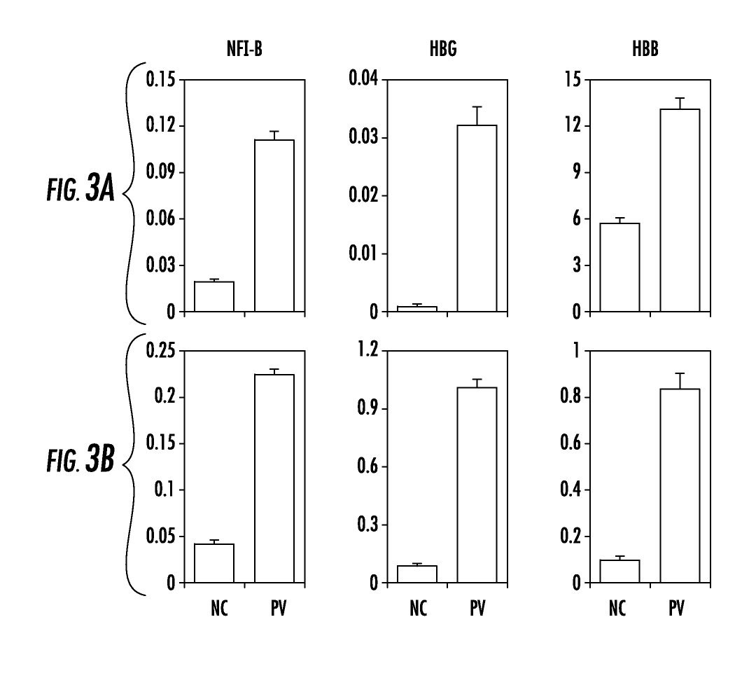

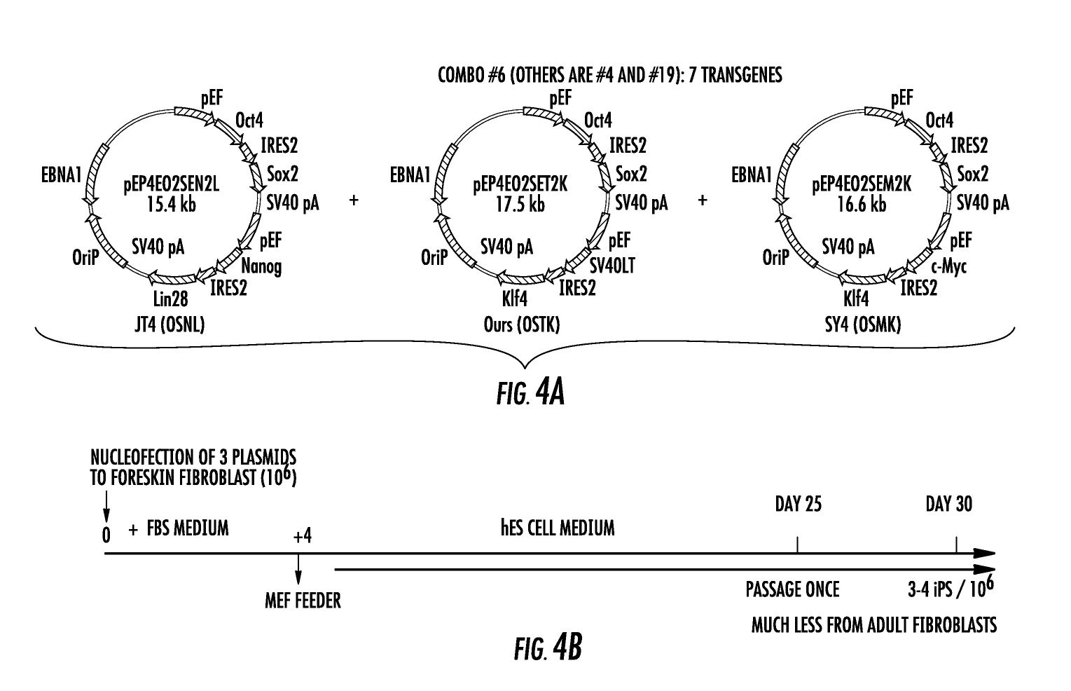

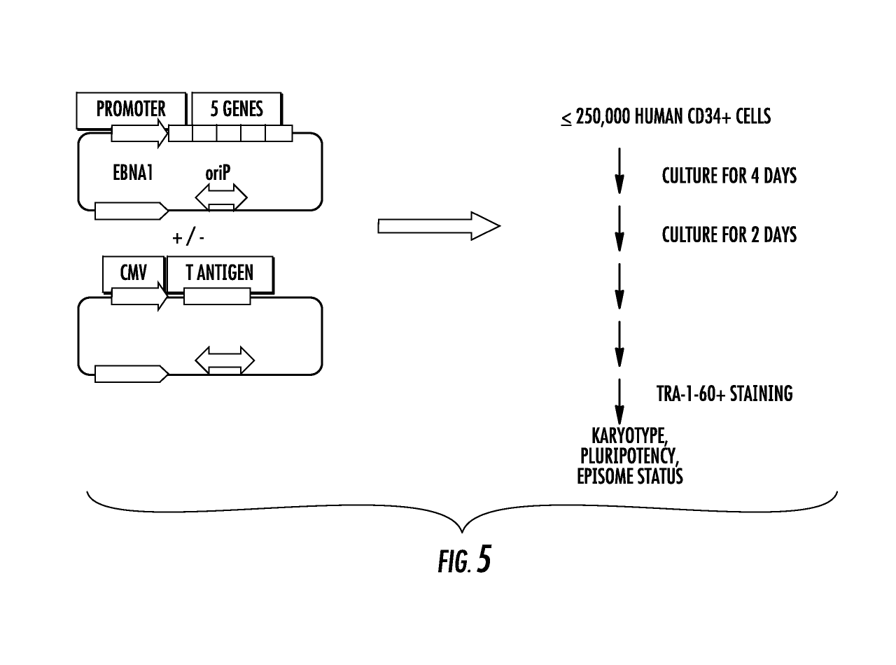

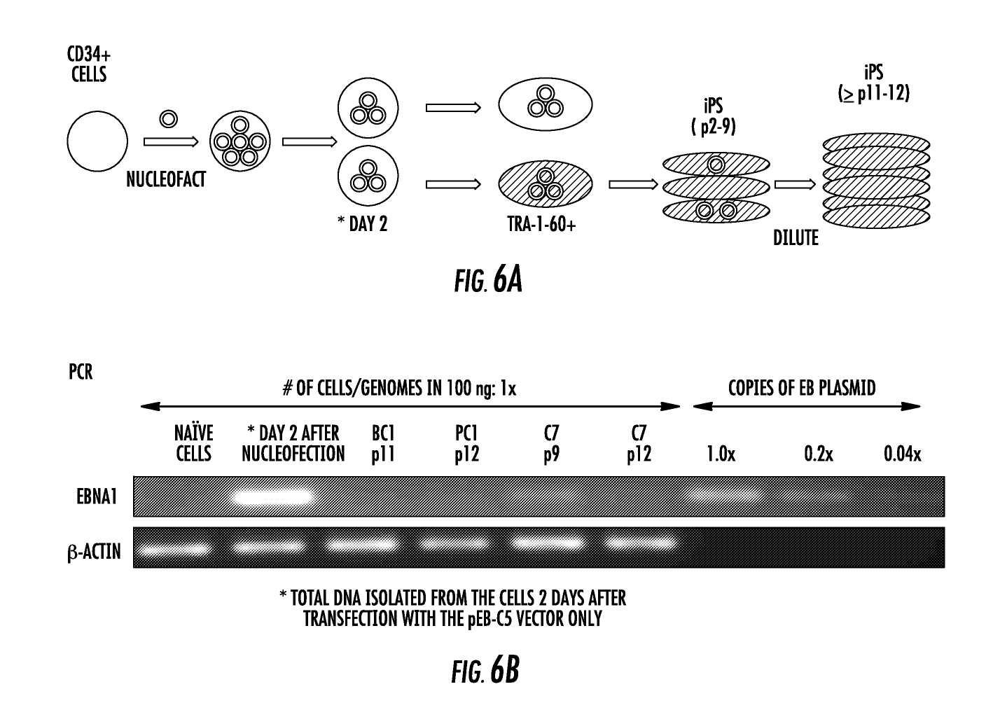

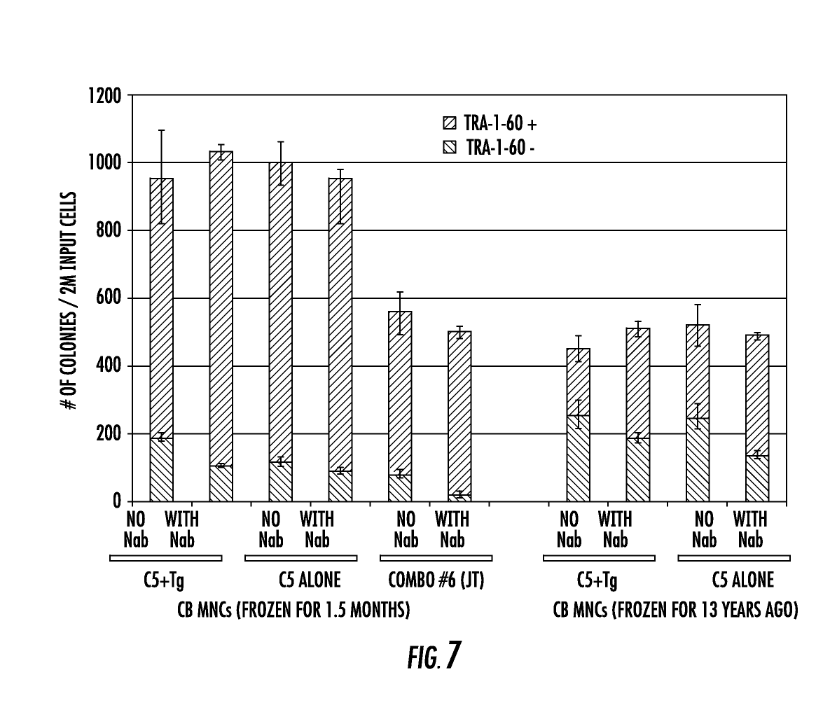

Although the PV iPS-derived hematopoietic progenitor (CD34+CD45+) cells can show a striking similarity in increased erythropoiesis (FIG. 2) and gene expression patterns (FIG. 3) to the primary CD34+ cells from the same PV patient when being compared to their respective normal controls, it is possible that only a few iPS clones can be used in full characterization of their hematopoietic potential. Additional iPS cell lines can be analyzed from more PV and other MPD patients and normal controls according to the invention. In addition, the invention provides the process of deriving iPS cell lines from the same patients using both blood cells (that carry the JAK2-V617F mutation) and marrow stromal cells (that lack the JAK2 mutation) (Mercier et al. (2009) Exp Hematol. 37:416-20), and their hematopoietic potential after the establishment of paired iPS cell lines can be compared according to the invention. In combination with the improving gene targeting technology in human iPS and ES cells, the invention provides iPS cell lines and their hematopoietic progeny as powerful tools to study how JAK2-V617F gene dosages and other genetic variations affect the MPD progenitor cell behavior (Zou et al. (2009) Cell Stem Cell 5:97-110).

One of the major concerns about the current iPS technology is the use and genomic integration of retroviruses. The invention provides improved methods that achieve reprogramming of human fibroblasts without permanent genome alteration will likely be applicable to that if human blood cells as well (Woltjen et al. (2009) Nature 458:766-70; Yu et al. (2009) Science 324:797-801; Yusa et al. (2009) Nat Methods 6:363-69; Zhou et al. (2009) Cell Stem Cell 4:381-84; Kim et al. (2009) Cell Stem Cell 4:472-76). These methods can allow us to derive adequate patient- or disease-specific iPS cells from blood cells for investigations of various blood diseases with either acquired or inherited mutations.

The invention also relates to unique epigenetic signatures of human blood cells permits efficient iPS cell derivation by a non-integrating plasmid. The invention provides efficient reprogramming of human adult peripheral blood (PB) or cord blood (CB) cells by 1-2 plasmids to generate induced pluripotent stem cells (iPSCs) with the unaltered genome. PB or CB mononuclear cells (MNCs) or purified CD34+ cells can be cultured for 4-9 days and then transfected once by novel EBNA1/OriP plasmids expressing reprogramming genes. Fourteen days after transfection by two plasmids, on average 250 and 9 iPSC-like colonies per 10⁶ transfected CB and PB CD34+ cells can be obtained, respectively. Alternatively, un-fractionated MNCs can be cultured for 8-9 days, and generated iPSCs by the same method. In one embodiment, a single EBNA1/OriP plasmid expressing 5 cellular factors can also generate iPSCs from all the cultured cell types, although the efficiency is ˜5-fold lower when adult PB cells are used. After reprogramming and expansion, the episomal DNA is gradually lost in proliferating iPSCs. These iPSCs have an unaltered nuclear genome and normal karyotype, and are pluripotent. The invention provides a method of generating integration-free human iPSCs by transfecting plasmids into briefly cultured and primed adult PB and CB MNCs, which show favorable epigenetic signature, will accelerate their use both in research and future clinical applications.

Human iPSCs that morphologically and functionally resemble human embryonic stem cells (ESCs) have been generated from many somatic cell types by viral vectors expressing defined reprogramming factors since 2007. Generation of human iPSCs from blood MNCs offers several advantages over other cell types (Yamanaka (2010) Cell Stem Cell 7(1):1-2). It is more convenient and less invasive to obtain PB than dermal fibroblasts and keratinocytes, which require several weeks to establish primary cell culture from skin biopsy. The inventor and others have successfully generated iPSCs from human immature MNCs expressing CD34 or CD133 markers from umbilical CB, adult PB and bone marrow (BM) by the standard retroviral vectors expressing 4 or fewer reprogramming genes after 2-4 day cultures (Ye et al. (2009) Blood 114:5473-80; Loh et al. (2009) Blood 113:5476-79; Giorgetti et al. (2009) Cell Stem Cell 5:353-57). Subsequently, 5 groups by using viral vectors generated human iPSCs from unfractionated MNCs, after longer cell culture to activate them into a proliferating state. Since T cells are most abundant in PB MNCs and easy to be expanded and infected by viral vectors, it is not surprising that all or the vast majority of the derived iPSCs in each of the 5 studies have a rearranged nuclear genome at the T cell receptor (TCR) locus.

Various virus-free and integration-free methods are currently being tested to reprogram human postnatal cells efficiently. However, most of these published studies using plasmids or proteins reported an extremely low efficiency, even when easiest cell types were used. Compared to other plasmid-based delivery methods such as the piggyBac DNA transposon and the mini-circle plasmids that elongate the expression and enable reprogramming human postnatal cells, the EBNA1/OriP episomal vector system which has been widely used in the past 2 decades offers several advantages. The EBNA1 trans element and OriP cis element derived from the EBV genome enables a simple plasmid to replicate and sustain as an episome in proliferating human cells. It can also persist episomally in human ESCs with little effect on their self-renewal and pluripotency. Episomal EBNA1/OriP plasmids delivered to human ESCs are lost gradually in the absence of any selection, likely due to epigenetic modification (such as DNA methylation) of the plasmid which leads to loss of EBNA1 expression and/or OriP functions.

Taking advantage of the unique properties of the EBNA 1/OriP plasmid, the Thomson/Yu group successfully reprogrammed human neonatal fibroblasts to integration-free iPSC lines (Yu et al. (2009) Science 324:797-801). However, the efficiency was low (˜3-6 per 10⁶ neonatal fibroblasts) even when 3 ENBA1/OriP plasmids expressing 7 genes were used. Subsequently, human fetal neural stem cells were shown reprogrammed by an epsiomal vector expressing fewer genes. For cell therapy development and disease modeling, however, it is highly desirable to develop an efficient method to generate integration-free and virus-free human iPSCs from adult somatic cells that are less permissive than their fetal/neonatal counterparts in reprogramming. The invention provides improved EBNA1/oriP plasmids to test if efficient reprogramming of postnatal somatic cells can be achieved by one poly-cistronic plasmid. In addition, the invention provides a better postnatal human cell type that are easy to obtain, prepare or prime for reprogramming, efficient to be transfected by plasmids, and more importantly, efficient to generate high-quality iPSCs.

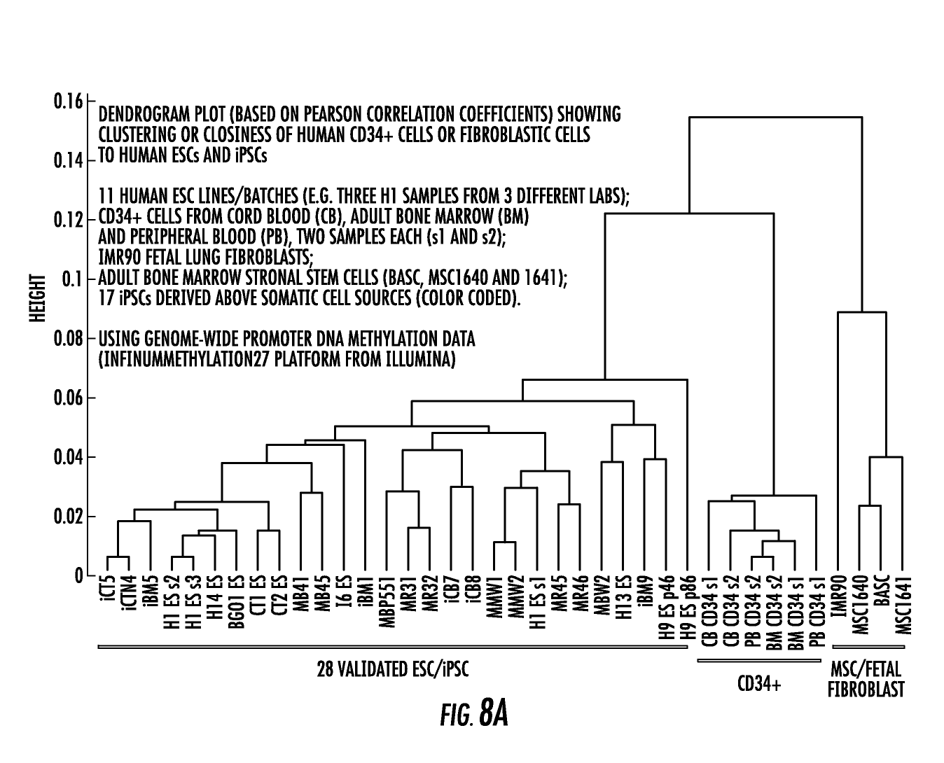

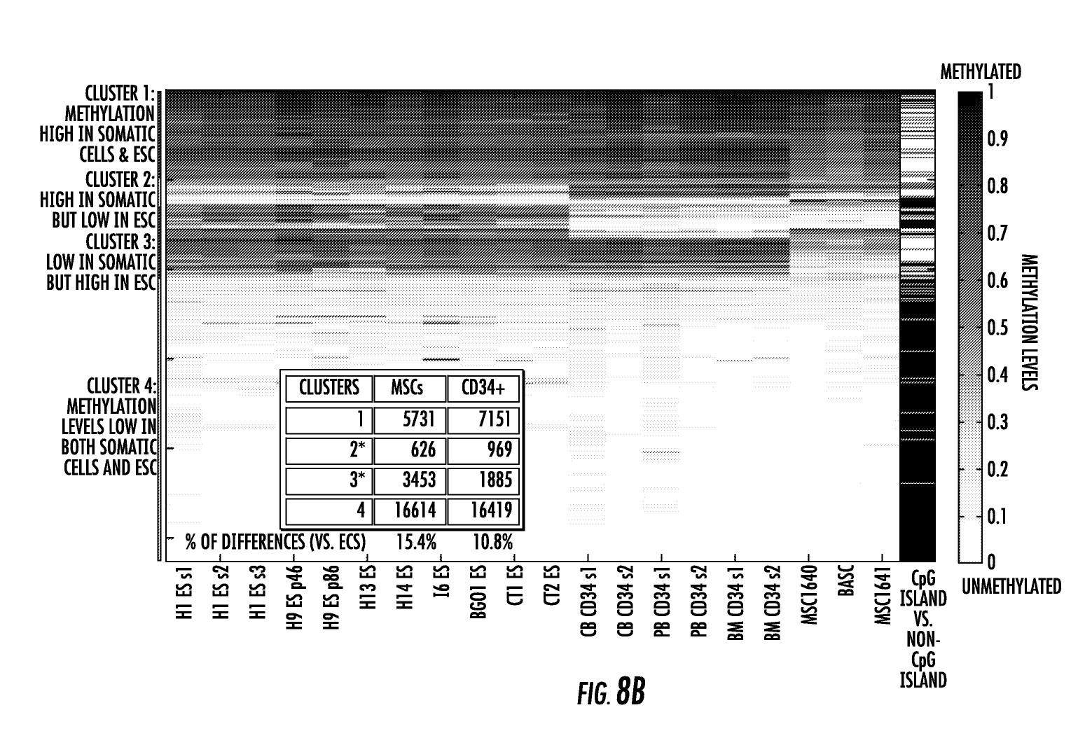

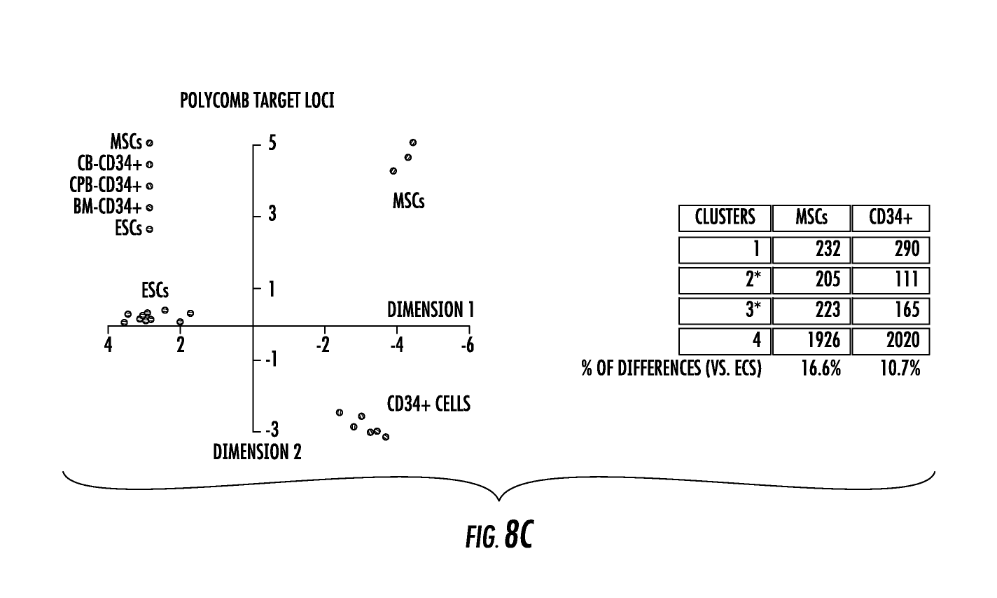

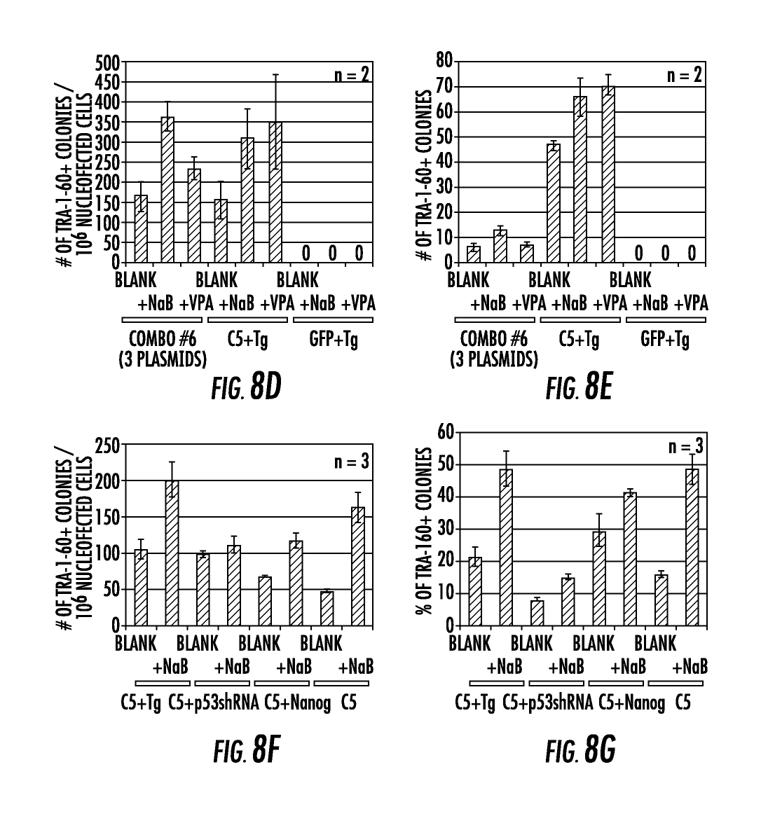

The observed higher efficiency with human CD34+ hematopoietic cells may result from a higher expression level of genes that are associated with or required for generating iPSCs and/or a higher proliferative activity under the culture conditions used. Not mutually exclusive, the high effieincy could be due to a favorable epigentic signature of immature human hematopoietic cells epcially after cell culture and activation by cytokines. The invention provides analysis of genome-wide promoter DNA methylation signatures of cultured human CD34+ cells from CB, adult PB and BM (2 samples each), cultured adult BM-derived MSCs (4 samples) and IMR90 fetal fibroblasts, and 17 iPSC lines derived from these somatic cells by various vectors. In addition, at least 11 human ESC samples to establish a baseline are included. Illumina's Infinium Methylation27 platform is used for DNA methylation of 27,578 informative CpG sites near the promoter region at single-nucleotide resolution, whose hypermethylation correlates with suppression of gene expression. Excluding the 1091 loci located in X and Y chromosomes, a dendrogram plot using Pearson correlation coefficients reveals that the 17 validated iPSC lines from various somatic cell sources are highly similar to ESCs, but distinct from their parental cells (FIG. 8A). The invention provides that cultured CD34+ cells cluster closer with the ESC/iPSC group, as compared to the MSCs/fetal fibroblast group.

To examine this feature more closely, the invention provides a K-means clustering analysis of the data (FIG. 8B). The levels of promoter DNA methylation (from 0 to 1) at 26,487 autosomal loci were analyzed. Four distinct clusters emerged, based on relative levels of promoter DNA methylation in somatic cells as compared to 11 ESCs as a baseline. Cluster #2 (high in somatic cells but low in ESCs, containing most pluripotency genes) and cluster #3 (low in somatic cells and high in ESCs) contain loci showing different promoter DNA methylation levels between somatic cells and ESCs. While 15.4% of loci in MSCs are different from ESCs, only 10.8% of loci in CD34+ cells are different from ESCs, suggesting that CD34+ HSPCs are closer to ESCs (and iPSCs, not shown) by this global analysis. When the inventor looks into genes that are hypermethylated in MSCs but hypomethylated in CD34+ cells and ESCs/iPSCs, the inventor notices the genes associated with pluripotency such as HMGA1 besides the CD34 marker gene (X, Y).

A novel set of ENBA1/OriP plasmids are used for reprogramming CB CD34+ cells with favorable epigenetic signatures. In the first EBNA1/OriP plasmid (called pEB-C5), 5 reprogramming factors (OCT4, SOX2, KLF4, c-MYC and LIN28) are expressed as a single poly-cistronic unit. In the second set of EBNA1/OriP plasmids, SV40 Large T antigen (Tg), NANOG or a small hairpin RNA targeting p53 (p53shRNA) is individually expressed. In the first set of 3 experiments, the pEB-C5 and pEB-Tg plasmids were used in comparison with the Thomson/Yu combination #6 containing 3 plasmids. After expansion (˜5-flod) with cytokines for 4 days, CB CD34+ cells were transfected once and then cultured following the standard protocol of deriving human iPSCs. The invention provides a procedure for iPSC generation, where isolated CB CD34+ cells were cultured with cytokines for 4 days, and then nucleofected by 2 or 3 plasmids (a total of 10 μg DNA) at day 0. Transfected cells were cultured for 2 more days before transferring onto 6 wells coated with MEF feeder cells. At day 3, the ESC culture medium was used in the presence or absence of NaB (0.25 mM) or VPA (0.5 mM). After day 9, the MEF-derived conditioned medium (CM) was used to substitute plain ESC medium. The Thomson/Yu combination #6 was more potent in generating transformed colonies than the 2-plasmid combination (pEB-C5+pEB-Tg). At day 14, cultures can be stained live with the TRA-1-60 antibody. TRA-1-60+ colonies with ESC-like morphology were picked after counting all the colonies. Most of colonies by combination #6, however, were not ESC-like even by day 10-14, as previously reported with neonatal fibroblasts. Few stained positive for TRA-1-60, a cell surface marker that is expressed in human ESCs and iPSCs.

It has been shown that acquisition of the cell surface expression of TRA-1-60 or a related antigen TRA-1-81 is a better marker for monitoring complete reprogramming of human somatic cells. The invention provides live staining of whole cultures at day 14 and numerated both TRA-1-60 positive and negative colonies. The 2-plasmid combination generated TRA-1-60+ colonies similar in numbers to combination #6 (FIG. 8D), but their percentages among total colonies were much higher (FIG. 8E). Sodium butyrate (NaB), a HDAC (histone deacetylase) inhibitor that stimulated reprogramming human fibroblastic cells, can also consistently enhance the number (and percentages) of TRA-1-60+ colonies by either vector combination (FIGS. 8D and 8E).

In the second set of experiments, the inventor replace Tg by either NANOG or p53shRNA, or omit Tg completely (FIGS. 8F and 8G). The overall efficiency of generating TRA-1-60+ colonies by the pEB-Tg plasmid added to the pEB-C5 plasmid is similar to that by an EBNA1/OriP plasmid expressing p53shRNA or NANOG (to a lesser extent), although p53shRNA generated more unwanted TRA-1-60- colonies. The pEB-C5 plasmid alone was able also to generate ˜50 TRA-1-60+ colonies from 10⁶ nucleofected cells (after 4 day expansion from ˜0.2×10⁶ original CD34+ cells). When pEB-C5 was used in the presence of with NaB during the reprogramming, ˜160 TRA-1-60+ colonies were generated (FIG. 8F).

After TRA-1-60 staining the inventor picked individual ESC-like colonies derived by transfection with either pEB-C5 alone (C), pEB-C5+ pEB-Tg (CT) or in the presence of NaB (CTN). At least two CB-derived clones from each category by standard assays are examined Undifferentiated phenotypes of one representative clone, CT5, can be observed. Undifferentiated phenotypes of iPSCs (clone CT5) derived from CB CD34+ cells (male) by the C5+Tg episomal vectors can be stained by various antibodies against selected markers. Immuno-fluorescence staining of OCT4, NANOG, SSEA4 as well as TRA-1-60 markers can show that the expanded CT5 clone display undifferentiated phenotypes unique to human ESCs/iPSCs. Additional clones such as CTN4 and C7 can also be examined in a similar manner. The invention provides that 5 of 6 clones have normal karyotypes (CT5 and CTN4). Karyotypes of iPSC clones CT5 and CTN4 (another representative clone derived by the C5+Tg vectors plus NaB) can be examined and shown a normal male karyotype. The normal karyotypes of two iPSC lines (C7 and CN1) derived by nucleofection of a single plasmid (pEB-C5) from a female sample can also be observed. Two out of two expanded iPSC clones derived by a single vector (pEB-C5) are also normal in karyotype and show undifferentiated phenotypes.

The invention provides genome-wide SNP analysis to examine the similarity of the CT5 and CTN4 genome to that of primary CB CD34− cells from the same CB donor. The genome-wide data (covering >10⁶ SNPs) indicate that CT5 and CTN4 iPSCs are essentially identical to CD34− cells of the CB donor and that reprogramming by the episomal vectors did not cause detectable alteration in the genome. The invention also provides analysis of the presence of the episomal DNA in reprogrammed cells at various stages. The invention provides PCR detection of episomal DNA using specific primers for EBNA1, Tg or beta-actin (genomic DNA). Un-transfected (naïve) cells or cells harvested at day 2 after the pEB-C5 transfection can be used as negative or positive controls, respectively. The pEB-Tg plasmid (containing both EBNA1 and Tg DNA) can be used as a common DNA control, in an amount equivalent to 1 or 0.2 copies per genome of cellular DNA. In general a trace amount of episomal DNA (<0.2 copies per cell) could be detectable after reprogramming (14 days) and expansion of 9 passages (˜50 days), but became undetectable by passage 11-12. The data are consistent with previous publications with neonatal fibroblasts or fetal neural progenitors that the EBNA1/OriP episomal DNA in human iPSCs is gradually lost after reprogramming

Pluripotency assay is also performed to iPSC lines derived with or without the transient expression of Tg (CT5, CTN4, and C7). The invention provides in vitro pluripotency test via embryoid body (EB) formation. Cell types derived from ectoderm (beta-3-tubulin), mesoderm (smooth muscle actin) and endoderm (alpha-fetal protein or AFP) can be found. The invention also provides in vivo pluripotency test by teratoma formation. Various cell types such as neural rosettes (ectoderm), cartilage (mesoderm) and glandular structures (endoderm) can be found. Pluripotency data of the CTN4 iPSC clone can be observed. Essentially all the 3 clones (CT5, CTN4 and C7) showed pluripotency indistinguishable from human ESCs and other validated iPSCs. The genome-wide promoter DNA methylation analysis of CT5 and CTN4 also shows that they are close to each other, and closely related to other iPSCs and ESCs (FIG. 8A). The same analysis also reveals DNA demethlyation at the promoter of pluripotency genes such as OCT4 in CT5 and CTN4 iPSCs.

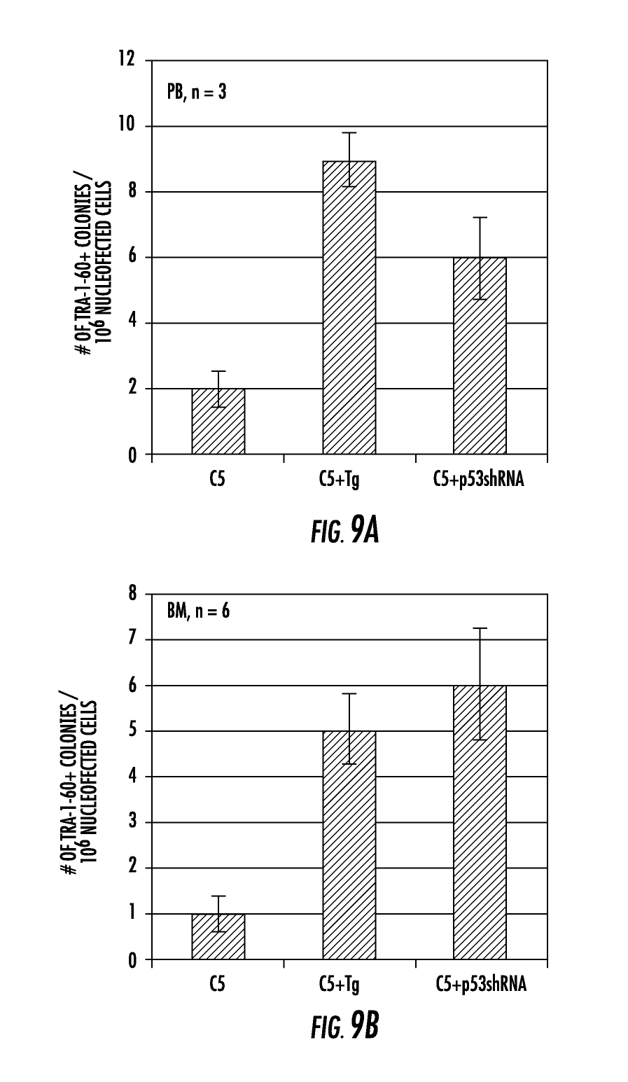

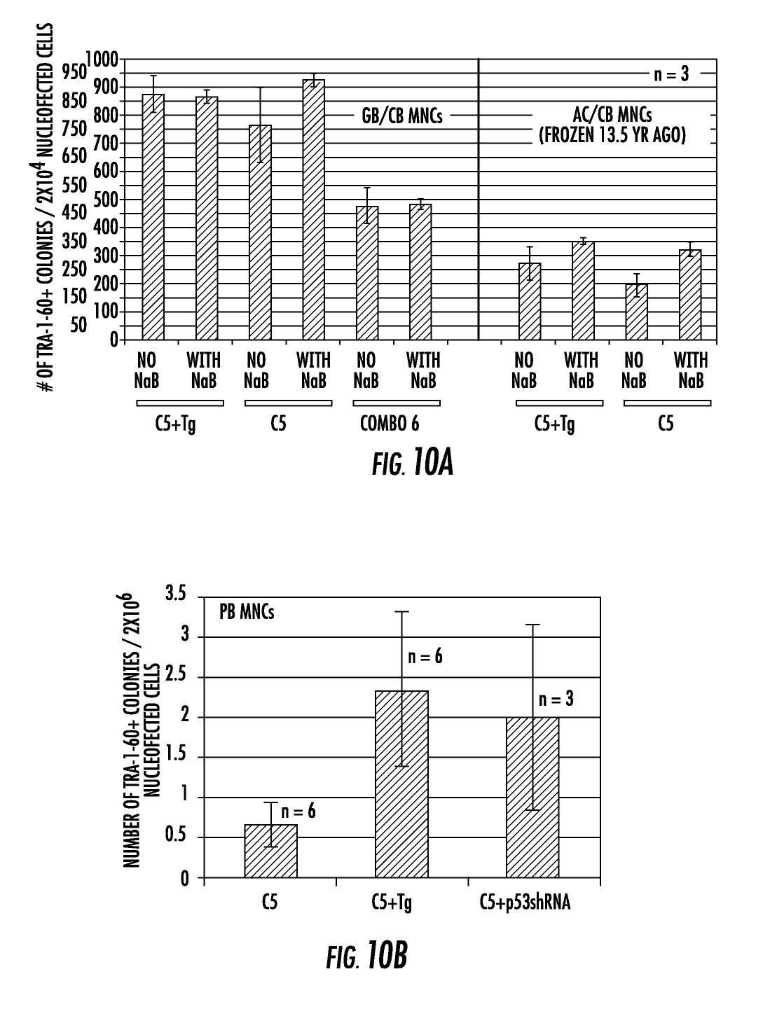

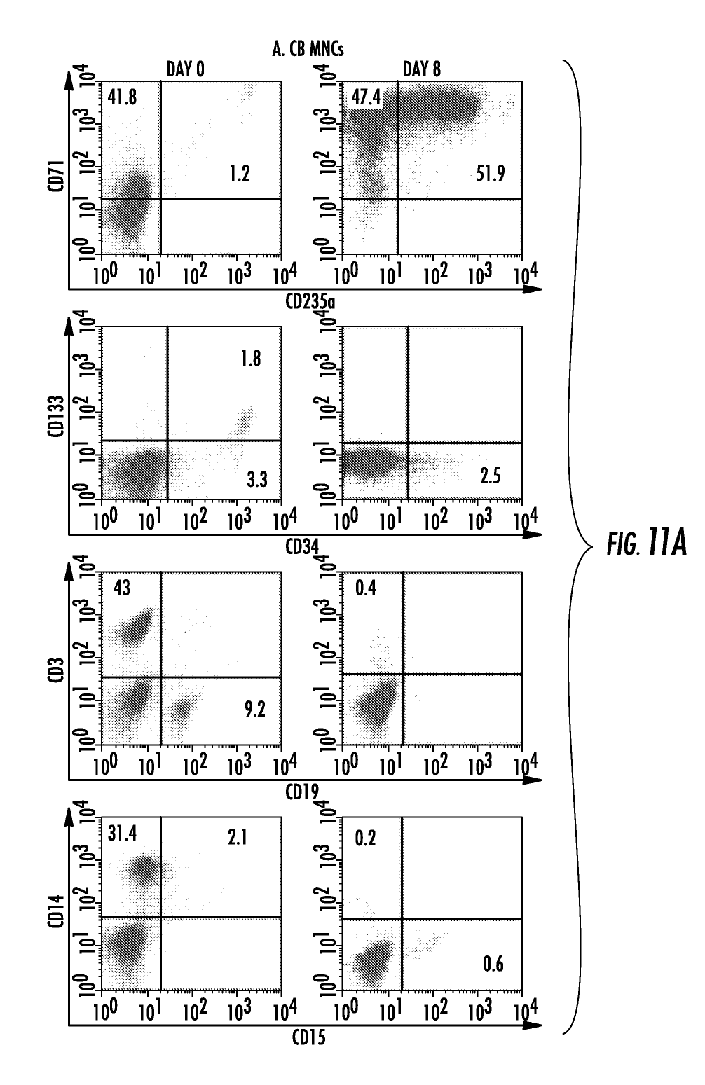

The invention provides an approach to derive iPSCs from human adult PB or BM CD34+ cells by 1 or 2 EBNA1/OriP plasmids. After 4 days of culture and expansion, 10⁶ primed cells were nucleofected by the same sets of plasmids. The ESC-like colonies emerged at day 10-12, about 4 days later than those from CB CD34+ cells. After live staining at day 14, the numbers of TRA-1-60+ reprogrammed colonies can be counted (FIGS. 9A and 9B). The efficiency of reprogramming adult CD34+ cells is ˜20-40 fold lower than CB CD34+ cells per 10⁶ cells transfected by the same vector sets. Human iPSC clones derived from adult CD34+ cells by the single pEB-C5 vector (i.e., without Tg) were picked and expanded as before. One clone from PB CD34+ cells (PC1) and one from BM CD34+ cells (BC1) were further characterized after 5-12 passages.

Human blood cells, especially their CD34+ fractions from CB, adult BM and PB that are enriched for HSPCs, showed an increased propensity to be reprogrammed by both retroviral vectors and plasmids than age-matched fibroblasts or adult BM-derived MSCs. This is likely due in part to the unique epigenetic signature in the CD34+ HSPCs. Three different methods of analyzing the genome-wide promoter DNA methylation data all point to a similar conclusion that human CD34+ HSPCs are more closely related to ESCs/iPSCs than adult MSCs (FIG. 8). However, these epigenetic differences are not sufficient to explain the 20-40 fold difference in reprogramming efficiency observed between the CB and adult PB CD34+ HSPCs. One possible factor is that CB CD34+ cells proliferate faster than adult CD34+ HSPCs, which will facilitate (epigenetic) reprogramming by cell division dependent mechanisms. The second possible factor for contributing to the higher efficiency of CB CD34+ cells would be ontogeny-related due to their neonatal origin. More epigenetic measurements and better DNA methylation coverage will enable to us to elucidate the molecular mechanisms that govern reprogramming or cell fate determination in general.

The EBNA1/OriP vector system of the invention offers several advantages over the previous vector combinations such as the combination #6. First, the pEB-C5 plasmid (+/−pEB-Tg) generated a higher percentage of TRA-1-60+ pre-iPSC colonies, especially in the presence of NaB, facilitating the isolation of desired TRA-1-60+ colonies. Second, the platform is more flexible if one needs to omit or replace Tg with other factors. The invention provides the pEB-C5 +/−pEB-Tg plasmid system in other adult somatic cell types such as adult MSCs, which have a low efficiency when the combination #6 vectors were used (<2 colonies per 10⁶ cells).

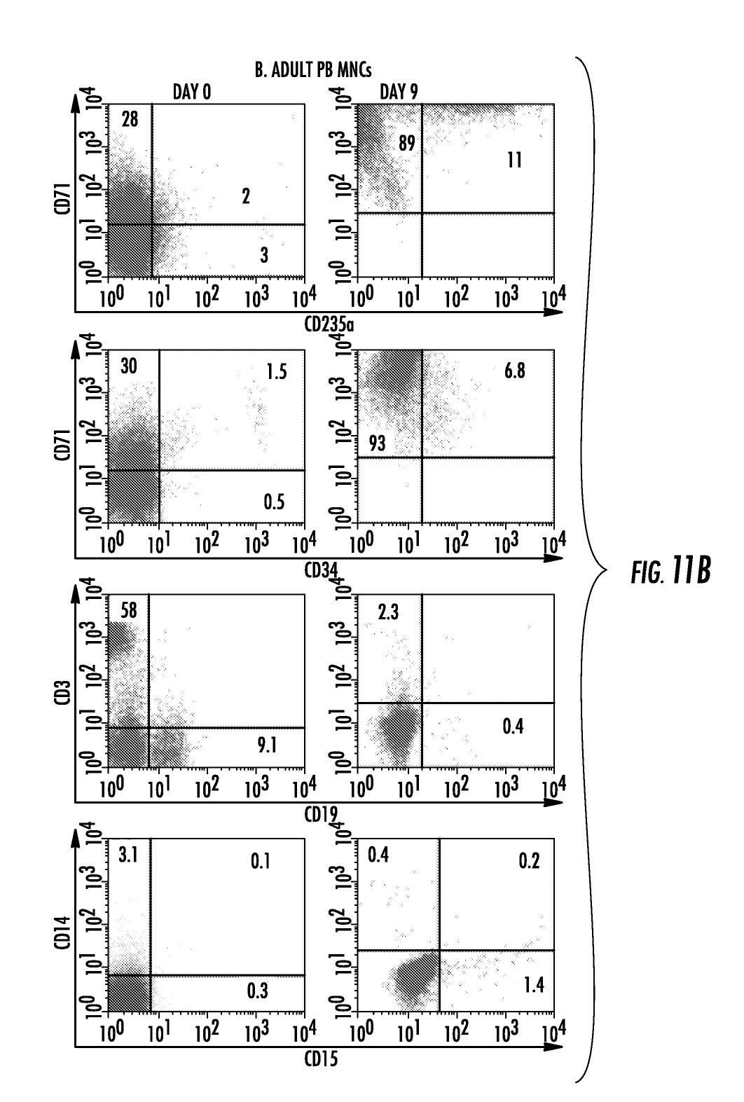

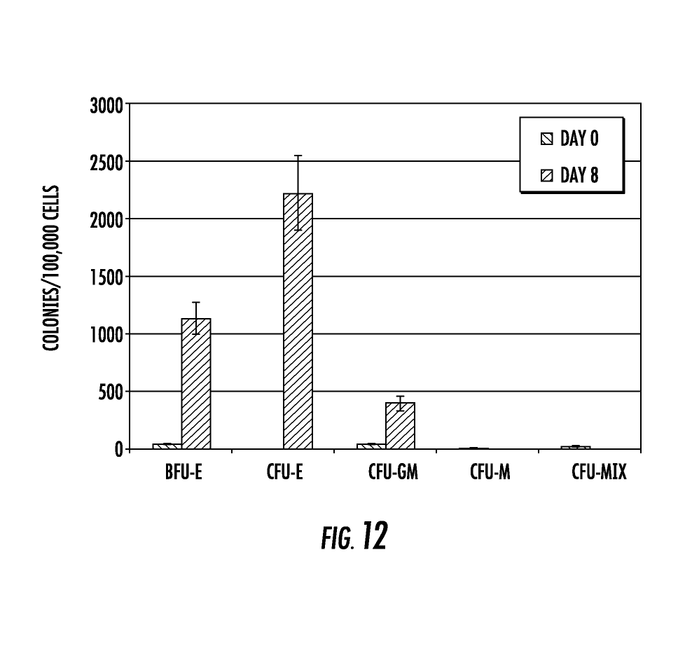

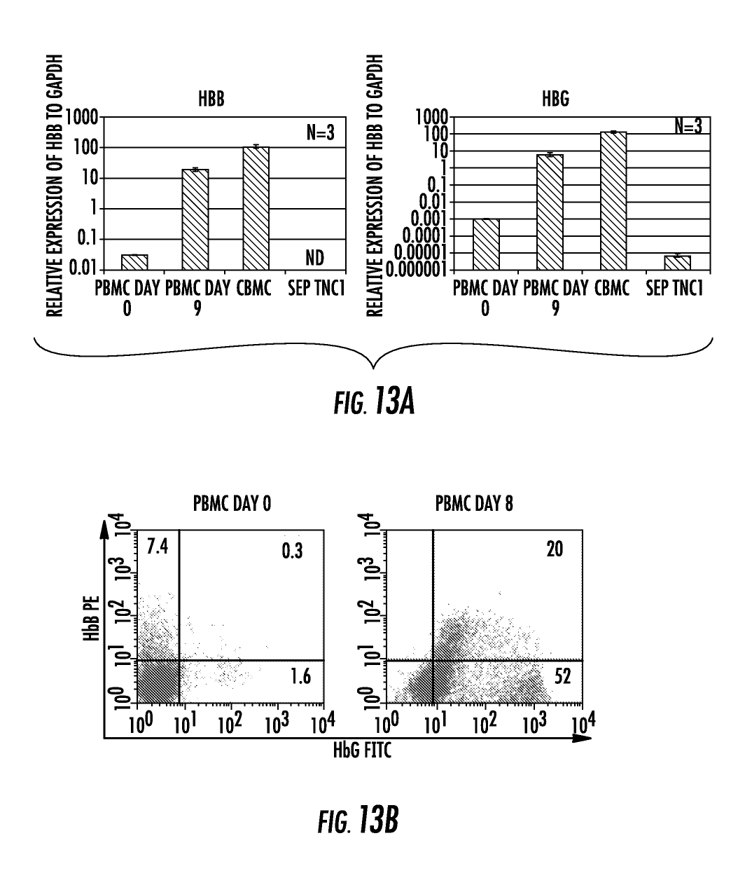

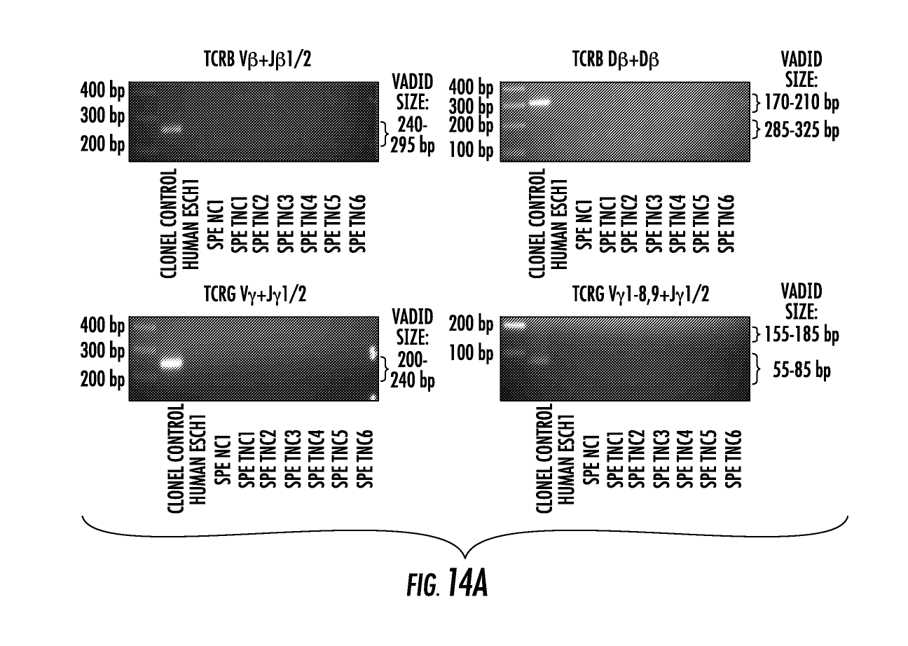

The flexible vector system of the invention allows direct comparison between the effects of Tg over-expression and knocking down p53 on reprogramming. Tg can block the functions of p53 and RB proteins that are major negative regulators of cell cycle progression, therefore promoting cell proliferation. The invention provides that the reprogramming efficiency is stimulated by 3-6 fold in human postnatal blood cells when transient expression of Tg or p53shRNA is used (FIGS. 9 and 10). Importantly, no genome alteration is observed. Therefore, the benefits (enhancing reprogramming efficiency) vs. risks (altering genomic integrity) of using Tg or p53shRNA are much more favorable when an episome-mediated transient expression is used. In certain embodiments, the invention can deliver 5-6 genes from a single ENBA1/OriP plasmid for transient expression, it is no longer critical how many factors are used.

The facile method of the invention to derive iPSC lines with the intact genome by using 1-2 plasmids provides several advantages over virus-mediated methods, including a possibly easier transition to generate clinical-grade iPSCs under GMP conditions in the future. Upon further improvements in the culture of un-fractionated PBMCs and a method to transfect fewer cells more effectively, the invention provides the ability to reprogram them more efficiently and consistently by the EBNA1/OriP plasmids. The efficient derivation of integration-free iPSCs from a small volume of blood will bring the iPSC technology to a new level.

In one embodiment, the invention relates to a method for reprogramming blood cells of a subject. The method includes introducing a plurality of factors into the blood cells, thereby reprogramming the blood cells to pluripotent or multipotent stem cells. In one aspect, a stable cell line is generated.

In one aspect, a viral DNA construct expressing a plurality of factors is introduced into the blood cells. In an additional aspect, the viral construct is a lentiviral construct or an adenoviral construct. In another aspect, the viral DNA construct includes a plurality of pluripotency genes operatively linked to at least one regulatory sequence for expressing the plurality of pluripotency factors. In another aspect, a non-viral construct is used to express the plurality of factors into the blood cells.

In another aspect, the method of the invention further includes adding at least one corticosteroid to the cells. In another aspect, the at least one corticosteroid includes a synthetic glucocorticoid. In another aspect, the at least one corticosteroid includes dexamethasone. In another aspect, the at least one corticosteroid is selected from the group consisting of aldosterone, beclometasone, betamethasone, cortisone, deoxycorticosterone, dexamethasone, fludrocortisone, hydrocortisone, methylprednisolone, prednisolone, prednisone, and triamcinolone.

In another aspect, the method further includes adding at least one cytokine to the cells. In one aspect, the at least one cytokine is selected from the group consisting of sertoli cell factor (SCF), Fms-related tyrosine kinase 3 ligand (FLT3 or FL), thrombopoietin (TPO), erythropoietin (EPO), and interleukin 3 (IL-3). In another aspect, the at least one cytokine includes sertoli cell factor (SCF), Fms-related tyrosine kinase 3 ligand (FLT3 or FL), and thrombopoietin (TPO). In another aspect, the at least one cytokine includes sertoli cell factor (SCF), erythropoietin (EPO), and interleukin 3 (IL-3).

In another aspect, the plurality of factors include pluripotency factors. In another aspect, the blood cells are cord blood (CB) cells, adult bone marrow (BM) CD34+ cells, adult peripheral blood (PB) cells, adult peripheral blood (PB) CD34+ cells, adult peripheral blood (PB) CD34+CD45+ cells, or adult peripheral blood mononuclear cells (PBMCs). In an additional aspect, the PBMCs are from a sickle cell anemia patient. In another additional aspect, the PBMCs include SCDB003 cells. In another aspect, the subject acquires a somatic mutation. In another aspect, the subject is a mammal In an additional aspect, the subject is human. In another aspect, the subject has myeloproliferative disorders (MPDs) and acquires JAK2-V617F somatic mutation.

In another aspect, the pluripotent or multipotent stem cells include hematopoietic stem cells. In another aspect, the plurality of factors include at least four factors selected from the group consisting of SOX2, SOX7, SOX17, OCT4, Nanog, LIN28, c-Myc, KLF4, ESRRB, EBF1, C/EBPα, C/EBPβ, NGN3, PDX and MAFA or active fragments thereof. In another aspect, the plurality of factors include OCT4, SOX2, KLF4, and c-MYC, or an active fragment thereof. In another aspect, the plurality of factors include OCT4, SOX2, KLF4, MYC, LIN28, and SV40 T antigen, or an active fragment thereof.

In another embodiment, the invention relates to a method for reprogramming blood cells of a subject. The method includes (a) introducing a first episomal vector into the blood cells; (b) generating stable cell lines from the cells of (a); and (c) losing the first episomal vector in the stable cell lines of (b) through a number of passages, thereby reprogramming the blood cells to pluripotent or multipotent stem cells.

In one aspect, the method further includes introducing a second episomal vector into the blood cells. In an additional aspect, the second episomal vector expresses SV40 T antigen (Tg). In another aspect, the first episomal vector includes a plurality of pluripotency genes operatively linked to at least one regulatory sequence for expressing a plurality of pluripotency factors. In an additional aspect, the episomal vector is a oriP/EBNA1 plasmid.

In another aspect, the method further includes adding at least one corticosteroid to the cells. In another aspect, the at least one corticosteroid includes a synthetic glucocorticoid. In another aspect, the at least one corticosteroid includes dexamethasone. In another aspect, the at least one corticosteroid is selected from the group consisting of aldosterone, beclometasone, betamethasone, cortisone, deoxycorticosterone, dexamethasone, fludrocortisone, hydrocortisone, methylprednisolone, prednisolone, prednisone, and triamcinolone.

In another aspect, the method further includes adding at least one cytokine to the cells of (a). In one aspect, the at least one cytokine is selected from the group consisting of sertoli cell factor (SCF), Fms-related tyrosine kinase 3 ligand (FLT3 or FL), thrombopoietin (TPO), erythropoietin (EPO), and interleukin 3 (IL-3). In another aspect, the at least one cytokine includes sertoli cell factor (SCF), Fms-related tyrosine kinase 3 ligand (FLT3 or FL), and thrombopoietin (TPO). In another aspect, the at least one cytokine includes sertoli cell factor (SCF), erythropoietin (EPO), and interleukin 3 (IL-3).

In another aspect, the blood cells are cord blood (CB) cells, adult bone marrow (BM) CD34+ cells, adult peripheral blood (PB) cells, adult peripheral blood (PB) CD34+ cells, adult peripheral blood (PB) CD34+CD45+ cells, or adult peripheral blood mononuclear cells (PBMCs). In an additional aspect, the PBMCs are from a sickle cell anemia patient. In another additional aspect, the PBMCs include SCDB003 cells. In another aspect, the subject is a mammal In an additional aspect, the subject is human. In another aspect, the pluripotent or multipotent stem cells include hematopoietic stem cells. In another aspect, the first oriP/EBNA1 expresses at least four factors selected from the group consisting of SOX2, SOX7, SOX17, OCT4, Nanog, LIN28, c-Myc, KLF4, ESRRB, EBF1, C/EBPα, C/EBPβ, NGN3, PDX and MAFA, or active fragments thereof In an additional aspect, the first oriP/EBNA1 expresses OCT4, SOX2, KLF4, Myc, and LIN28, or an active fragment thereof. In another aspect, the first episomal vector is undetectable after step (c) either as episomes or in the genome of the stable cell lines.

In another embodiment, the invention relates to a method of disease modeling. The method includes contacting the pluripotent or multipotent stem cells as described in any of the methods above with a test agent, and detecting a change in a function in presence of the test agent as compared to the function in absence of the test agent. In another embodiment, the invention relates to methods of generating subject-specific differentiated cells. The method includes inducing differentiation of the pluripotent or multipotent stem cells as described in any of the methods above, thereby obtaining a population of differentiated cells. In one aspect, the differentiated cells include blood cells, muscle cells, neuronal cells, connective tissues, or epithelial cells. In another aspect, the differentiated cells include blood cells. In an alternative aspect, the differentiated cells do not include blood cells. In another aspect, the differentiated cells include pancreatic beta cells, hepatocytes, cardiomyocytes, or skeletal muscle cells.

In another embodiment, the invention relates to a method of identifying an agent that alters a function of subject-specific differentiated cells. The method includes contacting the subject-specific differentiated cells as described in any of the methods above with a test agent, and detecting a change in a function in presence of the test agent as compared to the function in absence of the test agent, thereby identifying the test agent as an agent that alters a function of subject-specific differentiated cells. In one aspect, the function includes expression levels of at least one biomarker.

In another embodiment, the invention relates to a plurality of isolated pluripotent or multipotent stem cells reprogrammed from blood cells. In one aspect, the isolated pluripotent or multipotent stem cells are generated according to any of the methods described above. In another aspect, the isolated pluripotent or multipotent stem cells express a cell surface marker selected from the group consisting of: SSEA1, SSEA3, SSEA4, TRA-1-60, and TRA-1-81.

In another embodiment, the invention relates to a plurality of isolated subject-specific differentiated cells, wherein the subject-specific differentiated cells are generated according to any of the methods described above. In one aspect, the subject-specific differentiated cells include blood cells, muscle cells, neuronal cells, connective tissues, or epithelial cells. In an additional aspect, the subject-specific differentiated cells include blood cells. In an alternative aspect, the subject-specific differentiated cells do not include blood cells. In another aspect, the subject-specific differentiated cells include pancreatic beta cells, hepatocytes, cardiomyocytes, or skeletal muscle cells.

In another embodiment, the invention relates to a culture of undifferentiated pluripotent or multipotent stem cells, wherein the culture includes pluripotent or multipotent stem cells generated according to any of the methods described above. In another embodiment, the invention relates to a culture of subject-specific differentiated cells, wherein the culture includes subject-specific differentiated cells generated according to any the methods described above. In one aspect, the subject-specific differentiated cells include blood cells, muscle cells, neuronal cells, connective tissues, or epithelial cells. In another aspect, the subject-specific differentiated cells include blood cells. In an alternative aspect, the subject-specific differentiated cells do not include blood cells. In another aspect, the subject-specific differentiated cells include pancreatic beta cells, hepatocytes, cardiomyocytes, or skeletal muscle cells.

In another embodiment, the invention relates to a method of treating a disease requiring replacement or renewal of cells. The method includes administering to a subject an effective amount of the pluripotent or multipotent stem cells generated according to any of the methods described above. In another embodiment, the invention relates to a method of treating a disease requiring replacement or renewal of cells. The method includes administering to a subject an effective amount of the differentiated cells generated according to any of the methods described above.

In another embodiment, the invention relates to a reprogramming non-viral episomal vector. The non-viral episomal vector includes at least four pluripotency genes operatively linked to at least four regulatory sequences for expressing at least four pluripotency factors. In one aspect, the non-viral episomal vector includes a oriP/EBNA1 plasmid. In another aspect, the non-viral episomal vector is a oriP/EBNA1 plasmid. In one aspect, the non-viral episomal vector includes at least five pluripotency genes operatively linked to at least five regulatory sequences for expressing at least five pluripotency factors. In another aspect, the non-viral episomal vector includes at least six pluripotency genes operatively linked to at least six regulatory sequences for expressing at least six pluripotency factors. In another aspect, the non-viral episomal vector includes at least seven pluripotency genes operatively linked to at least seven regulatory sequences for expressing at least seven pluripotency factors.

In another embodiment, the invention relates to a method of enhancing reprogramming efficiency of cells. The method includes adding at least one corticosteroid to the cells. In another embodiment, the invention relates to a method of enhancing generation of pluripotent or multipotent stem cells. The method includes adding at least one corticosteroid to the cells. In one aspect, the at least one corticosteroid includes a synthetic glucocorticoid. In another aspect, the at least one corticosteroid includes dexamethasone. In another aspect, the at least one corticosteroid is selected from the group consisting of aldosterone, beclometasone, betamethasone, cortisone, deoxycorticosterone, dexamethasone, fludrocortisone, hydrocortisone, methylprednisolone, prednisolone, prednisone, and triamcinolone.

In one aspect, the method further includes adding at least one cytokine to the cells. In one aspect, the at least one cytokine is selected from the group consisting of sertoli cell factor (SCF), Fms-related tyrosine kinase 3 ligand (FLT3 or FL), thrombopoietin (TPO), erythropoietin (EPO), and interleukin 3 (IL-3). In another aspect, the at least one cytokine includes sertoli cell factor (SCF), Fms-related tyrosine kinase 3 ligand (FLT3 or FL), and thrombopoietin (TPO). In another aspect, the at least one cytokine includes sertoli cell factor (SCF), erythropoietin (EPO), and interleukin 3 (IL-3).

The following examples are provided to further illustrate the advantages and features of the present invention, but are not intended to limit the scope of the invention. While they are typical of those that might be used, other procedures, methodologies, or techniques known to those skilled in the art may alternatively be used. All citations are incorporated by reference unless indicated otherwise.

### EXAMPLES

### Example 1

### Human CD34+ Cells and Reprogramming by Gene Transduction

Culture media and conditions for expanding human ES cells and iPS cells: Media and culture conditions for derivation, expansion and karyotyping (G banding) of human iPS cells are described previously (Mali et al. (2008) Stem cells 26:1998-2005).

Frozen human CD34+ cells from CB and adult BM are purchased from AllCells and Poietics (now part of Lonza). Previously frozen PB CD34+ cells from two patients registered at the Johns Hopkins Center of Chronic MPDs (Moliterno et al. (2008) Exp Hematol. 36:1480-86) are also used in this study and from whom written informed consent was obtained. Isolated PB CD34+ cells after G-CSF mobilization are purchased from AllCells and used as a normal control for analyzing gene expression. Four classic retroviral vectors pMXs-Oct4, pMXs-Sox2, pMXs-Klf4 and pMXs-c-Myc encoding (mouse) reprogramming factors constructed by the laboratory of Dr. Yamanaka are obtained from Addgene (www.addgene.org).

Retroviral supernatants are produced by transfection of 293T cells with a mixture of three plasmids: one transducing (reprogramming) vector, a plasmid expressing the VSV-G envelope protein and a helper plasmid expressing the retroviral Gag/Pol gene. After thaw, CD34+ cells are cultured for 2-4 days with cytokines SCF (100 ng/ml), FL (50-100 ng/ml) and TPO (20 ng/ml) before retroviral transduction. Stimulated CD34+ cells (2×10⁵) are mixed with the retroviral supernatants supplemented with 4 ng/ml polybrene, and cultured with the same medium and the three cytokines. After 2-3 days of transduction and following culture, transduced cells at day 5 post transduction are plated at a density of 4×10⁵ cells /well in 6-well plates. They are cultured on pre-seeded embryonic fibroblast (MEF) feeder cells for programming similar to previously described (Mail et al. (2008) Stem Cells 26:1998-2005). At day 7, the hES cell medium is used and throughout.

### Example 2

### Immuno-Staining of Undifferentiated iPS Cells and their Derivatives

TRA-1-60 live staining: TRA-1-60 antibody (Millipore, 1:300) and Alexa555-conjugated secondary antibody anti-Mouse IgM (Invitrogen, 1:400) are diluted in hES medium and added into reprogramming plate. The plate is incubated in 37° C. for 1 hour before medium is changed to fresh conditioned medium. TRA-1-60 positive colonies are identified under an inverted fluorescence microscope.

Immuno-staining of iPS clones for undifferentiated markers and of differentiated cells after embryoid body (EB) formation are performed as previously described (Mail et al. (2008) Stem Cells 26:1998-2005; Chen et al. (2008) Cell Stem Cell 2:345-55; Yu et al. (2008) Cell Stem Cell 2:461-71).

### Example 3

Teratoma Formation Assay of Pluripotency

Three to five million iPS cells are harvested by Collagenase IV (Sigma) digestion, washed with PBS and resuspended in 200 μL diluted (1:1) Matrigel solution. Cells are injected intra-muscularilly into Rag1-/-γC-/- mice or other improved immuno-deficient mice with a further reduced level of natural killer cells. Tumors are excised 6-10 weeks after injection. Histological processing is performed as previously described (Mail et al. (2008) Stem Cells 26:1998-2005; Chen et al. (2008) Cell Stem Cell 2:345-55; Yu et al. (2008) Cell Stem Cell 2:461-71). Teratoma RNA is extracted using Trizol reagent (Invitrogen) according to manufacturer's recommendation. RT-PCR of AFP, CD34, PAX6, OCT4 and NANOG human genes was carried out as previous described (Mail et al. (2008) Stem Cells 26:1998-2005; Chen et al. (2008) Cell Stem Cell 2:345-55; Yu et al. (2008) Cell Stem Cell 2:461-71).

### Example 4

### DNA Fingerprinting and JAK2V617F Allele Analysis

Genomic DNA isolation and DNA fingerprinting using Invitrogen's MapPairs primers for PCR are performed as described. JAK2 allele analysis is carried out using a custom TaqMan SNP Genotyping Assay (Applied Biosystems) for JAK2-V617F as previously described (Moliterno et al. (2006) Blood 108:3913-15; Moliterno et al. (2008) Exp Hematol. 36:1480-86).

### Example 5

### Differentiation and Data Analysis

Hematopoietic differentiation and analysis: The hematopoietic differentiation potential of CD34+ cell derived-iPS cells are examined by an improved method of EB formation (so called spin-EB) and hematopoietic differentiation under a feeder- and serum-free condition, similar to what previously described (Yu et al. (2008) Cell Stem Cell 2:461-71; Ng et al. (2005) Blood 106:1601-03; Ng et al. (2008) Nat Protoc. 3:768-76). The EBs are harvested between week 2-3 and are analyzed by hematopoietic colony-forming assays and FACS for the presence of hematopoietic markers as previously described (Yu et al. (2008) Cell Stem Cell 2:461-71; Zhan et al. (2004) Lancet 364:163-71). Cytospin is conducted as previously described (Zhan et al. (2004) Lancet 364:163-71) and the slides were stained with Hema 3 Stain Set (Fisher Diagnostics).

Erythroid differentiation and expansion in liquid culture: Two to 3 weeks after the spin-EB hematopoietic differentiation, the CD34+CD45+ cell populations are collected using FACS sorting from age matched normal iPS EBs and PV-iPS EBs. 2×10⁴ CD34+CD45+ cells are cultured in serum free medium containing SCF (20 ng/ml), IL-3 (50 ng/ml) and EPO (1 U/ml). Cells are harvested on day 7 and the corresponding cell numbers are counted. The differentiated cells are also stained with anti-CD235a antibody (BD Biosciences) and anti-CD45 antibody (Invitrogen) for FACS analysis.

Data presentations and statistics: For flow cytometric (FACS) analysis, at least 10,000 events are collected and analyzed. The percentages of selected cell populations (based on the comparison with background staining shown when isotype-matched antibodies are used) among the total live cells are shown. For histogram presentations, mean and standard derivation (SD) are shown. The Wilcoxon two-sample test is applied when the number of replicates (n) is small (n<10). This test is run unsupervised with the SAS 9.1 package (SAS Institute, Cary, N.C., USA). Findings are judged to be statistically significant if p was 0.05 or less.

### Example 6

### Quantitative Real-Time PCR Analysis for Gene Expression

After EB-mediated hematopoietic differentiation (21 days), CD34+CD45+ cells from both normal BM CD34+ cells-derived iPS and MPD183 CD34+ cells-derived iPS are sorted and used for RNA extraction using Trizol reagent following manufacturer's protocol (Invitrogen). cDNA is prepared using random hexamers and quantitative PCR is performed with primers and probes for Nuclear Factor I-B (NFI-B), hemoglobin-gamma (HBG), and hemoglobin-beta (HBB) genes supplied by Applied Biosystems. Real-time PCR is conducted using the 7500 Real-Time PCR System (Applied Biosystems). The human leukemia cell line DAMI is used to generate a standard curve. Individual gene expression signals are normalized to beta-actin in the same cell sample.

### Example 7

### Reprogramming of Cord Blood and Adult CD34+ Cells

To test whether postnatal human blood cells could be reprogrammed by the conventional four reprogramming factors, CD34+ cells purified from CB and adult BM that show extensive proliferative potential in culture are tested (Novelli et al. (1999) Hum Gene Ther. 10:2927-40). After thawing, the CD34+ cells are cultured and activated for 2-4 days to stimulate cell proliferation (Novelli et al. (1999) Hum Gene Ther. 10:2927-40), before gene transduction by the 4 standard retroviral vectors (Takahashi et al. (2007) Cell 131:861-72). Colonies resembling human ES/iPS cells emerge ˜day 16 after gene transduction from CB samples and ˜day 21 from adult BM CD34+ cells. One week later, the whole culture is stained live with the TRA-1-60 antibody recognizing a cell-surface epitope on undifferentiated hES and iPS cells. The invention provides that live culture can be stained by the TRA-1-60 antibody, 3-4 weeks after transduction of CB CD34+ cells. TRA-1-60+ colony can be first seen at week 3 and picked at week 4 (after re-staining)

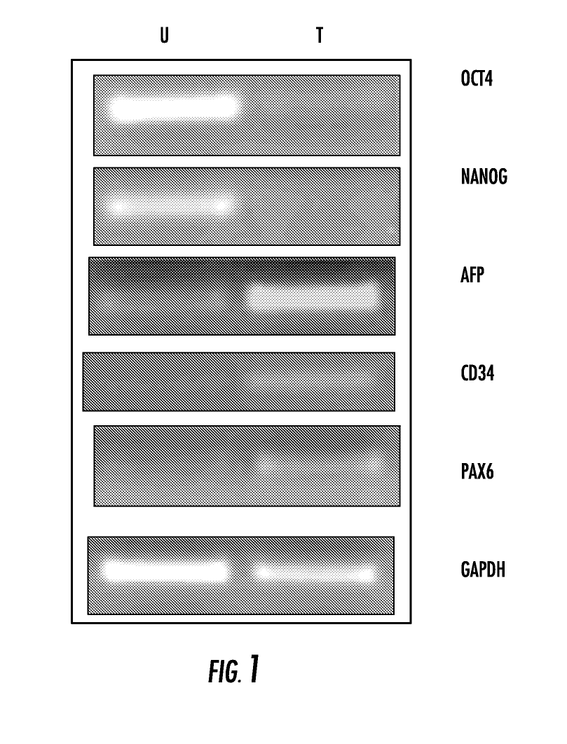

The invention provides that TRA-1-60 staining is also a convenient and reliable way of identifying candidate iPS colonies derived from human blood cells. Various colonies 4 weeks after transduction of BM CD34+ cells can be identified, after live staining with TRA-1-60 and a secondary fluorescent reagent. A smaller fraction of formed colonies are TRA-1-60+. TRA-1-60+ colonies are individually picked and give rise to iPS clones. After expansion, the picked TRA-1-60-positive clones can show the characteristic hES/iPS cell morphology and the expression of other pluripotency markers in addition to TRA-1-60. Immuno-fluorescence staining images of expanded iPS cells from CB can be observed. In addition to TRA-1-60, they also express other pluripotency markers including NANOG and SSEA4. They no longer express CD34 or CD45 markers, and instead express OCT4 and NANOG genes from the endogenous loci (FIG. 1).

After expansion for ≧9 passages, multiple iPS lines examined retain a normal karyotype. Pluripotency of these iPS clones is demonstrated by differentiation assays such as in vitro embryoid body (EB) formation and in vivo teratoma formation, generating various cell types derived from the three embryonic germ layers. Differentiation potential of CB-derived iPS cells after in vitro differentiation by embryoid body (EB) formation (10 days) can be observed, where AFP staining for endoderm, CD34 staining for mesoderm, and b-III tubulin staining for ectoderm can be observed. In addition, in vivo differentiation potential after teratoma formation from CB derived iPS cells can be observed. Hematoxylin and Eosin staining of various slides after sectioning can show various tissues from the three embryonic germ layers: gut epithelium (endoderm), cartilage (mesoderm) and glycogenated epithelium (ectoderm). After immuno-fluorescence staining, differentiated cells expressing AFP, CD34 and β-III-tubulin (an ectoderm marker) can be seen. The image of specific staining is overlaid by DAPI staining of nuclei. Therefore the iPS cells derived from human CB and adult BM CD34+ cell lines resemble both hES cells and fibroblast-derived iPS cells morphologically, phenotypically and functionally.

Human iPS cell lines derived from MPD patient peripheral blood cells: Next CD34+ cells isolated from the peripheral blood of two MPD patients (without G-CSF mobilization) are tested (Moliterno et al. (2008) Exp Hemotal. 36:1480-86). An acquired common somatic mutation in the JAK2 gene (1849G>T, resulting in a V617F substitution that activates the intracellular kinase) is present in the patients' blood cells as in >95% of PV, and ˜50% of ET and PMF patients (James et al. (2005) Nature 434:1144-48; Kralovics et al. (2005) N. Engl J Med. 352:1779-90; Levine et al. (2005) Cancer Cell 7:387-97; Zhao et al. (2005) J. Biol. Chem. 280:22788-92; Baxter et al. (2005) Lancet 365:1054-61; Moliterno et al. (2006) Blood 108:3913-15; Levine and Gilliland (2008) Blood 112:2190-98; Skoda R. (2007) Hematology Am Soc Hemotol Educ Program 1-10). Both MPD patients (MPD 183 with PV and MPD562 with PMF) have a heterozygous JAK2-V617F genotype in 100% of colony-forming erythroid progenitors in their PB CD34+ cells (Moliterno et al. (2008) Exp Hematol. 36:1480-86). Using the same quantitative allele-specific PCR analysis (Moliterno et al. (2006) Blood 108:3913-15; Moliterno et al. (2008) Exp Hematol. 36:1480-86), the invention provides that the percentage of the mutated (1849T resulting in V617F) JAK2 allele in the thawed CD34+ cells from each patient is ˜50% (Table 1).

Applying the same reprogramming protocol used for normal human CD34+ cells, the invention provides multiple iPS clones from each of the two patients (Table 1). All of the iPS clones successfully expanded are heterozygous for JAK2-V617F, identical to the parental CD34+ cells (Table 1). Similar to human iPS cells derived from fibroblasts and normal CD34+ cells, the expanded JAK2-V617F iPS clones can display the characteristic undifferentiated hES/iPS cell morphology and marker expression Immuno-staining of different colonies from a representative iPS line (Clone 8) derived from MPD183 shows the expression of undifferentiated cell markers TRA-1-60, SSEA4 and NANOG. Analysis of differentiated cells after EB formation and teratoma formation reveal the presence of various cell types originated from the three embryonic germ layers, indicating pluripotency of these patient-specific iPS lines. The invention provides a pluripotency test for the iPS Clone 8 from MPD183 (iMPD183.C8) after EB formation (day 10) as previously for human ES cells and other (normal) iPS cells, showing that JAK2-V617F iPS cells can also differentiate into various cell types expressing markers of three embryonic germ layers. Similar results can be obtained from the iPS Clone 3 of the second MPD patient (iMPD562.C3) before and after EB-mediated differentiation. The invention also provides in vivo differentiation potential after teratoma formation from MPD183 derived iPS cells. Hematoxylin and Eosin staining of various slides after sectioning can show various tissues from the three embryonic germ layers: gut epithelium (endoderm), cartilage (mesoderm) and glycogenated epithelium (ectoderm). The expanded JAK2-V617F iPS cell lines from both MPD patients also can show a normal karyotype by G-banding analysis. Expanded iPS lines from the two female MPD patients (MPD183 and MPD562) can be observed to retain a normal karyotype (46,XX), after 10 and 11 passages, respectively.

Directed hematopoietic differentiation of blood cell-derived iPS cells: The hematopoietic differentiation potential of human iPS cell lines derived from normal and PV CD34+ cells is examined by an improved method of EB formation and differentiation under a feeder- and serum-free condition (Yu et al. (2008) Cell Stem Cell 2:461-71; Ng et al. (2005) Blood 106:1601-03; Ng et al. (2008) Nat Protoc. 3:768-76). By week 2, many small cells resembling immature hematopoietic cells grow out of the EBs. Human iPS cells derived from normal control (NC) adult CD34+ cells or the PV CD34+ cells can be plated in microtiter wells and aggregated for EB formation and directed hematopoietic differentiation. Approximately after 10-14 days, substantial numbers of small round cells resembling immature hematopoietic cells surrounding EBs can be found and increase in next several days. The cells are subsequently harvested and analyzed by both hematopoietic colony-forming assays and FACS for the presence of hematopoietic markers. Total cells are subsequently harvested and assayed for the presence of hematopoietic markers and of hematopoietic colony-forming units (CFUs) formed in semi-solid methylcellulose media (Normal control iPS and PV-iPS). CFU-granulocyte/ monocyte and CFU-erythroid colonies can be observed after additional 10-14 days in culture. A similar CFU assay using purified from CD34+CD45+ cells from a NC and PV sample is also performed. The invention provides Wright-Giemsa staining after cytospin of individually picked myeloid and erythoid colonies generated from iPS cells derived from normal CD34+ cells. Cells resembling erythroblasts and multiple lineages of myeloid cells can be observed. Both myeloid and erythroid colonies can be detected as previously using hES cells (Zhan et al. (2004) Lancet 364:163-71). This is confirmed when the purified CD34+CD45+ cells generated from differentiated iPS cells are used, which are derived from either normal or the PV CD34+ blood cells. Staining of individual cells from picked hematopoietic colonies confirms the presence of various myeloid and erythroid cell types.

TABLE 1
JAK2-V617F genotyping of iPS cell lines derived from MPD patients.

| | % Wild-type | % V617F allele |
|---|---|---|
| Cells Analyzed | allele (1849G) | (1849T) |
| CD34+ cells from MPD183 | 48 | 52 |
| iPS MPD183 clone 1 | 46 | 54 |
| iPS MPD183 clone 2 | 45 | 55 |
| iPS MPD183 clone 3 | 48 | 52 |
| iPS MPD183 clone 5 | 46 | 54 |
| iPS MPD183 clone 6 | 45 | 55 |
| iPS MPD183 clone 7 | 46 | 54 |
| iPS MPD183 clone 8 | 46 | 54 |
| iPS MPD183 clone 10 | 47 | 54 |
| iPS MPD183 clone 11 | 50 | 50 |
| CD34+ cells from MPD562 | 50 | 50 |
| iPS MPD562 clone 2 | 46 | 54 |
| iPS MPD562 clone 3 | 49 | 51 |

In addition, FACS analysis confirms the presence of differentiated hematopoietic cells at 13-17 days after the EB formation. CD45+ (27%-64%) and CD43+ (36%-60%) hematopoietic cells expressing undetectable to intermediate levels of CD34 marker are also observed. Gene expression analysis by RT-PCR can also show the up-regulation of hematopoietic markers RUNX1, GATA-1, GATA-2 and HBB genes as well as the down-regulation of the pluripotency-specific marker NANOG gene. The invention provides that the iPS cells derived from normal and MPD blood CD34+ cells can also be re-directed to multiple hematopoietic cell lineages (Loh et al. (2009) Blood 113:5476-79).

Enhanced erythropoiesis of hematopoietic progenitor cells generated from PV iPS cells: A principal feature of PV is overproduction of red blood cells, which is recapitulated by increased erythropoiesis of purified PB PV CD34+ hematopoietic progenitor cells in vitro. Using the standard colony-forming assay that measures both myeloid and erythroid progenitor cells, the invention provides that purified (CD34+CD45+) hematopoietic progenitor cells generated from the PV-iPS cells after EB-mediated hematopoietic differentiation can form more erythroid colonies than from the normal control. To further analyze their erythropoiesis potential, the purified CD34+CD45+ cells differentiated from both iPS cell lines are cultured in two types of media containing EPO, SCF and IL-3, the conditions favoring erythropoiesis from CD34+ progenitor cells (FIG. 2) (Ugo et al. (2004) Exp Hematol. 32:179-87; Dupont et al. (2007) Blood 110:1013-21).

After 7 days in a liquid culture, both normal control and PV cell populations can display extensive cell proliferation (FIG. 2A). However, the proliferation rate with the PV sample is about twice that of the normal control (FIG. 2A), recapitulating the increased erythropoiesis using primary CD34+ cells from the corresponding PV patient. To ensure that enhanced cell proliferation observed in PV-iPS cells is associated with erythroid commitment and further differentiation, the invention provides analysis for the expression of CD235a (also known as Glycophorin A, a specific marker for erythroid differentiation and maturation) and CD45 (a pan-leukocyte marker that is also expressed in progenitor cells but down-regulated after erythroid differentiation). While 35% of the cells in the normal control group can show the erythroid phenotype (CD235a+CD45−), 53% of the cells in the PV sample can display the erythroid phenotype (FIG. 2B), indicating the enhanced erythroid differentiation as well as proliferation.

A similar enhanced erythropoiesis in the PV sample can be observed when the purified CD34+CD45+ cells generated from differentiated iPS cells are grown in a serum and methylcellulose-containing medium. After 14 days of culture, there is a near two-fold proliferation advantage in the PV sample over the normal control (FIG. 2C). Flow analysis can also show a greater percentage of cells expressing the erythroid phenotype (FIG. 2D). PV-iPS generated hematopoietic progenitor cells show a gene expression pattern similar to the primary CD34+ cells from the PV patient: Whole-genome microarray analysis is conducted on purified CD34+ cells from PV patients and normal healthy donors in an effort to identify differentially expressed genes in PV patients. In addition to Nuclear Factor I-B (NFI-B) (Kralovics et al. (2002) Exp Hematol. 30:229-36) and hemoglobin-gamma (HBG) (Papayannopoulou et al. (1979) Blood 53:446-54) genes that have been previously reported, hemoglobin-beta (HBB) gene expression is also found up-regulated in CD34+ cells of PV patients including MPD183. The microarray data of the MPD183 (PV) patient can be further confirmed using real-time quantitative PCR analysis of the 3 genes (FIG. 3A). Notably, the expression pattern can be also observed in the CD34+ cells generated from the PV-iPS cell line that is derived from the corresponding PV patient (FIG. 3B). Together, the data shown in FIGS. 4 and 5 demonstrate that hematopoietic progenitor cells generated from differentiated PV-iPS cells recapitulate principal features of increased erythropoiesis of the primary hematopoietic progenitor cells isolated from the corresponding PV patient.

### Example 8

### Generating Human iPS Cells Using Non-Integrating Plasmids

Human induced pluripotent stem cells (iPSCs) that are functionally similar to embryonic stem cells (ESCs) hold great potential for cell and gene therapies, disease modeling and drug development. The earliest success was achieved by using adherent fibroblastic cells and retroviral vectors that transduce fibroblasts very efficiently. It is also highly desirable to reprogram postnatal blood cells, including those from cord blood (CB) and adult peripheral blood (PB), which are easily accessible and less exposed to environmental mutagens. In 2009, reprogramming of human postnatal blood cells have been achieved using the 4 Yamanaka factors delivered by retroviral vectors. Provided are experiments where reprogramming efficiencies of CB and PB CD34+ cells can be higher than age-matched fibroblasts or MSCs. This may result from an epigenetic profile of hematopoietic CD34+ cells that appears closer to iPSCs/ESCs than that of fibroblasts/MSCs to iPSCs/ESCs.

To generate integration-free iPSCs that produce hematopoietic progeny efficiently, OriP/EBNA1 episomal vectors are used to reprogram adult PB as well as CB cells, which were used previously to reprogram foreskin fibroblasts albeit at a low efficiency. When one of the best combinations (#6, 3 plasmids) is used, 1-3 candidate iPSC clones per 1 million cells can be obtained. The efficiency of generating iPS clones is even lower with human adult somatic cells by the 3 vectors. To improve the reprogramming efficiency, a new episomal reprogramming vector system using 1-2 OriP/EBNA1 plasmids is constructed. One (pEB-C5) expresses 5 factors (OCT4/SOX2/KLF4/Myc/LIN28), and the second expresses SV40 T antigen (Tg). CB and adult PB CD34+ cells are first cultured for 4 days and expanded ≧4-folds. The expanded cells (1 million) are then transfected once by the 1-2 new OriP/EBNA1 plasmids constructed according to the invention. Fourteen days later, on average 250 and 9 TRA-1-60+, iPSC-like colonies from CB and adult PB cells can be obtained, respectively, when both pCB-05 and pEB-Tg are used. A single plasmid (pEB-C5) can also generate iPSCs although the efficiency is ˜4-folds lower. Five characterized iPSC lines derived from CB and adult PB CD34+ cells (with or without Tg) are karyotypically normal and pluripotent.

After successful reprogramming and expansion, episomal DNA is gradually lost in proliferating iPSCs. After serial expansions for 11-12 passages, vector DNA is undetectable either as episomes or in the genome of the 5 iPSC lines. The invention also provides embodiments to reprogram un-fractionated adult PB mononuclear cells (PBMCs) including those from a sickle cell patient (SCDB003). To achieve better cell proliferation that is critical to iPSC production, a culture condition that favors the formation and proliferation of erythroblasts from PBMCs is used. PBMCs purified by standard Ficoll gradient are cultured in a serum-free condition with cytokines SCF, EPO and IL-3. Although cell death is observed and cell number decreases significantly in the first 4 days, equal or more cells than input can be obtained by day 8. The expanded cells morphologically resemble pro-erythroblast cells, and express high-level CD71. Less than 1.5% of them express markers of T cells (CD3, CD2, CD4 and CD8) and B cells (CD19 and CD20).

When 2×10⁶ expanded SCDB003 cells (achievable from PBMCs in 1 ml or less PB) are transfected by the 2 OriP/EBNA1 plasmids and reprogrammed in the presence of butyrate, the invention provides 8 colonies at day 14 that are TRA-1-60+ and iPSC-like. The second plasmid (pEB-Tg) is not essential although it can enhance the efficiency by ˜4 folds. 3 iPSC-like colonies derived from PBMCs with or without Tg are picked and characterized. All of them express pluripotency markers and behave as typical iPSCs. No evidence shows if they are derived from committed T or B cells that somatic mutations altered and rearranged their genomes. The invention also provides embodiments examining karyotypes, in vivo pluripotency, and status of episomal vectors in 3 PBMC-derived iPSCs. As compared to recent studies using viruses that preferentially reprogram human T cells with a rearranged genome, the invention is virus-free and genomic alteration-free using 1-2 plasmids. The ability to obtain integration-free human iPSCs from a few ml PB by 1-2 plasmids can greatly accelerate uses of iPSCs in both research and future clinical applications, epically for blood disease modeling and treatment.

### Example 9

### Human CD34+ Cell Culture and Priming for Reprogramming by Episomal Vector Transfection

Culture Media and Conditions for Expanding Human iPSCs, and Karyotyping: Most of human primary hematopoietic mononuclear cells (MNCs) obtained from anonymous donors were collected and frozen at AllCells, LLC (Alameda, Calif.), including human MNCs expressing a high-level of the CD34 surface marker (CD34+) from newborn cord blood (CB), adult bone marrow (BM) and peripheral blood (PB) after G-CSF mobilization. CD34+ cells were isolated at AllCells or by the inventor using the CD34 MACS beads (Miltenyi, Auburn, Calif.), although CD34-depleted MNCs also contain MNCs expressing a low-level CD34 surface expression. For selected cord blood and peripheral blood samples donated by the parents or patients via their doctors, the inventor isolates MNCs using a standard protocol by Ficoll-Paque Plus (p=1.077) purchased from GE HaelthCare.

The human ESCs and established iPSCs were maintained on a mitotically inactivated mouse embryonic fibroblast (MEF) feeder layer (GlobalStem or Millipore) in KNOCKOUT/DMEM medium (Invitrogen) supplemented with 20% KNOCKOUT Serum Replacement (KSR) (Invitrogen), 2 mM L-glutamine (Invitrogen), 2 mM nonessential amino acids (Invitrogen), 1× antibiotic/antimycotic mix (Invitrogen), 0.1 mM β-mercaptoethanol (Sigma), and 10 ng/ml basic fibroblast growth factor (bFGF). All cytokines were purchased from Peprotech if not otherwise indicated. Karyotyping of human ESCs and iPSCs by G-banding (300-500 bands) was examined by a certified cyto-geneticist by the method previously described (Ye et al. (2009) Blood 114:5473-80; Mali et al. (2010) Stem Cells 28:713-20).

Constructions of Episomal Vectors: All transgenes were cloned into the EBNA1/OriP-based pCEP4 episomal vector (Invitrogen) for reprogramming The cDNA for the SV40 large T antigen was PCR cloned and inserted into the pCEP4 plasmid backbone as previously into a lentiviral vector. Other vectors expressing other reprogramming transgenes individually were constructed with the cDNAs for the open reading frames (ORFs) of human NANOG, OCT4, SOX2, LIN28 obtained by direct PCR of human ESC H9 cell cDNA. Other cDNA templates (human or mouse) were from existing plasmids. The expression cassette for expressing p53-shRNA was obtained by digesting a fragment from a pSUPER-based vector, and inserted into the pCEP4 plasmid. The pSUPER-shRNA plasmid was purchased from Addgene (Cambridge, Mass.). A single EBNA1/OriP vector expressing five (mouse) factors Oct4, Sox2, K1f4, c-Myc and Lin28 linked by 2A sequences and under the control of the synthetic CAG promoter was constructed in the pCEP4 plasmid backbone, similar to a piggyBac transposon plasmid previously used (Mali et al. (2010) Stem Cells 28:713-20). The 3 EBNA1/OriP plasmids used in the combination #6 for neonatal fibroblasts (Yu et al. (2009) Science 324:797-801) were also purchased from Addgene and used in transfection at the reported optimal ratio.

Frozen human CD34+ cells from cord blood (CB), adult bone marrow (BM) and peripheral blood (PB) after G-CSF mobilization were purchased from AllCells. After thawing, CD34+ cells were cultured for 4 to 5 days under a serum-free medium (SFM): containing 50% IMDM with 50% Ham's F12 (Invitrogen), ITS supplement (insulin-transferrin-selenium and 5 mg/ml BSA), synthetic lipids, 50 ug/ml of ascorbic acid and 2 mM glutamine (all from Sigma). The SFM is further supplemented with cytokines: SCF (100 ng/ml), FL (100 ng/ml), TPO (20 ng/ml) and IL-3 (10 ng/ml) for expanding CD34+ cells, similar to previously described (Ye et al. (2009) Blood 114:5473-80). For reprogramming with EBNA 1/OriP-containing episomal vectors, combinations of plasmids (up to 10 μg total) were co-transfected into 1×10⁶ human CD34+ cells via nucleofection (Human CD34 Cell solution [Nucleofector Kit VPA-1003] with U-008 program, Amaxa/Lonza).

Transfected CD34+ cells were cultured in one well of a 12-well plate in the same medium and cytokines for another 2 days. Subsequently, transfected cells were transferred to 3 to 6 wells of MEF-coated 12-well plates and cultured in MEF medium (DMEM+10% FBS). Plates were spun at 500 rpm for 30 min at room temperature to help cells attach to MEF-coated plates. The next day, MEF medium was replaced with the ESC medium. Small organic molecules that enhance reprogramming like sodium butyrate (NaB, 0.25 mM) and Valproic Acid (VPA, 0.5 mM) were also added at this time when indicated (15). Culture medium was exchanged every other day. Starting day 9 post-transfection, MEF conditioned medium (CM) was used to sustain the development of colonies. Colonies with morphology similar to iPSC colonies were readily visible on day 6 to 10 post-transfection of CB CD34+ cells, and on day 10 to 14 post-transfection of adult BM and PB CD34+ cells.

### Example 10

### Human MNC Culture and Priming for Reprogramming by Episomal Vector Transfection

After thawing, frozen MNCs from CB and PB) were cultured in the SFM supplemented with the following cytokines and a hormone: SCF (50 ng/ml), IL-3 (10 ng/ml), EPO (2U/ml, R&D Systems), IGF-1 (40 ng/ml) and dexamethasone (1 uM, Sigma). The media were replenished at day 3 and 6. By day 8-9 when an overt sign of cell division was observed, cells were harvested for nucleofection as well for various analyses. Approximately 3.5-fold cells were obtained for CB at day 8 and 1.5-fold for PB MNC at day 9. After optimizing nucleofection of the cultured MNCs to achieve maximal cell survival and gene transfer, the inventor settled down with the same Human CD34 Cell solution [Nucleofector Kit VPA-1003] but with 2×10⁶ cultured cells. The transfected MNCs were cultured in the same medium for days and then further reprogrammed on MEF feeder cells as described above with cultured CD34+ cells.

Although integration-free iPSCs from ˜0.2×10⁶ adult PB and CB CD34+ cells can be generated by a single EBNA1/OriP plasmid (pEB-C5) and one-time transfection, it is still highly desirable for many applications to reprogram from unfractionated blood MNCs without CD34 cell isolation. The CD34+ cell culture (with 4 cytokines SCF, IL3, FLT3 ligand and TPO) might not sufficiently stimulate CB or PB MNCs to proliferate and massive cell death occurred after 4 days. Un-cultured (containing 1%-2% CD34+ cells) or the remaining MNCs harvested after the CD34+ cell culture did not result in iPSC generation after nucleofection by the two plasmids (C5+Tg) that generated 200-360 iPSCs from 10⁶ expanded CB CD34+ cells in parallel.

By testing various culture conditions that stimulate MNC proliferation, the invention provides one condition that expands erythroblasts or red blood progenitor cells, not on those expanding T and/or B cells undergone somatic mutations that alter the genome. Under the serum-free condition with SCF, IL3, EPO, IGF-1 and Dex that are known to stimulate erythroblast proliferation, the inventor observes the majority of cells proliferating and resembling erythroblasts after 8-9 day culture of frozen or freshly isolated CB and or PB MNCs. Despite cell death and number reduction in the first several days, 2×10⁶ MNCs (present in 0.25-2 ml blood volume) gave rise to 7.5×10⁶ cells by day 8 for CB and 2-3×10⁶ MNCs by day 8-9 for PB MNCs. In addition to cell proliferation, the cultured PB MNC cells expressed fetal as well as adult hemoglobin (HBG and HBB; FIG. 13), indicating they are distinct from uncultured primary cells in patterns of gene expression and/or epigenetic signatures.

The invention provides a condition to efficiently transfect 2×10⁶ cultured and activated MNCs (8-9 days) by nucleofection with the same 1-2 EBNA1/OriP episomal vectors. Following the same protocol, the invention provides that the cultured CB MNCs can efficiently generate TRA-1-60+ colonies. Although variations of different CB MNCs (frozen 1.5 months or 13.5 years ago) existed, the overall reprogramming is very high (930 and 320 iPSCs per 2×10⁶ MNCs) by the single pEB-C5 episomal vector (FIG. 10A). The stimulatory effect of Tg or NaB is minimal under the culture condition here. The generation of iPSCs by the 1-2 EBNA1/OriP episomal vectors was also achieved from PB MNCs from an adult sickle cell patient (SCD003), although the efficiency is much lower (FIG. 10B). Similar to adult PB CD34+ cells, Tg (or blocking p53 by shRNA) increased the efficiency by 3-4 fold. The pEB-C5 vector is sufficient to generate iPSCs although NaB-dependent reprogramming efficiency is lower (0.6 per 2×10⁶ MNCs). Because it is easy to obtain 2×10⁶ MNCs (from 0.5-2 ml blood volume), the efficiency (0.6-2.4 iPSCs) is adequate for most applications.

### Example 11

### PCR Analysis of Episomal Vectors

Total DNA from cell extracts was isolated from cells using DNeasy® blood and tissue kit (Cat. No. 69506, Qiagen). 50 ng DNA isolated from different cellular samples was used as a template for the 30-cycle PCR reaction. All the PCR reactions were carried out with Taq DNA polymerase (Invitrogen). Two sets of primers used for detecting plasmid DNA (in either episomal or integrated form) are: EBNA1_D (SEQ ID NO:1): 5′-TTTAATACGATTGAGGGCGTCT-3′, EBNA1_U (SEQ ID NO:2): 5′-GGTTTTGAAGGATGCGATTAAG-3′, Tg_F (SEQ ID NO:3): 5′-GCCAGGTGGGTTAAAGGAGC-3′, Tg_R (SEQ ID NO:4): 5′-GGTACTTATAGTGGCTGGGCTGT-3′. The pEB-Tg plasmid was used as a control template, in an amount diluted equivalent to 1, 0.2 or 0.04 copies per genome of 50 ng genomic DNA. The beta-actin detection by PCR was used to normalize the amount of DNA used for PCR. The total DNA from isolated naive CD34− cells (AllCells) were used as negative control while the sample from CB CD34+ cells transfected with pEB-C5 plasmid (day 2 post-transfection) were used a positive control for EBNA1 transgene detection.

### Example 12

### Embryoid Body (EB) Formation, Differentiation and Immuno-Staining

TRA-1-60 Live Staining for identifying fully reprogrammed clones: TRA-1-60 antibody (mouse IgM, used as 1:300 of MAB4360, Millipore) and Alexa555-conjugated anti-mouse IgM secondary antibody (1:500, Invitrogen) were diluted in ESC medium and added into reprogramming cell culture plates. The cell culture plates with sizable ESC-like colonies were incubated in 37° C. for 1 hour before medium was changed to fresh ESC medium or CM. TRA-1-60 positive colonies were identified under an inverted fluorescence microscope, and can be picked up within 1 day after staining for further expansion and characterization.

Immuno-staining of Undifferentiated iPSCs: To characterize clones by immuno-staining of various markers, cultured human iPSCs were fixed by 4% paraformaldehyde in PBS for 15 min, followed by permeabilization (required for detecting intracellular antigens) by 0.1% Triton X-100 for 15 min, then washed with PBS for 3 times. The fixed samples were incubated with the following primary antibodies for 2 hours at room temperature: anti-TRA-1-60 (1:300), anti-SSEA-4 (1:10, MC-813-70, mouse IgG, Developmental Studies Hybridoma Bank, Iowa City, Iowa), anti-NANOG (1:100, 1 μg/ml, rabbit IgG, Peprotech), anti-OCT4 (1:100, sc-5279, mouse IgG, Santa Cruz Biotechnology). After a brief wash with PBS, Alexa555 conjugated goat anti-rabbit or anti-mouse secondary antibodies (1:500, Invitrogen) were used for one-hour incubation to visualize the cells together with DAPI nuclear staining. In order to visualize the alkaline phosphatase (AP) activity, iPSCs were fixed and stained with Sigma's FAST BCIP/NBT (B5655, Sigma). The AP assay was performed as per the manufacturer instructions.

Embryoid Body (EB) Formation: EBs were formed using differentiation medium similar to culture medium except withdrawing bFGF and replacing 20% KSR with 20% FBS. Human iPSCs (near confluent) from 2 wells of a 6-well plate were used for EB formation and cultured in one well of ultra-low attachment 6-well plate. After 8 days, EBs were transferred to gelatin-coated 24-well plates for additional 2-days of attachment. Immuno-staining of EBs is similar to that of human ESCs or iPSCs, with the following primary antibodies used: mouse-anti-β3-tubulin (1:1000, Sigma), mouse anti-actin (Sigma), smooth muscle isoform (1:500, CBL171, Millipore), rabbit anti-AFP (1:500, DAKO). Alexa 555 conjugated goat anti-mouse or anti-rabbit secondary antibodies (1:500, Invitrogen) were used for visualization.

The invention provides undifferentiated phenotypes of adult CD34+ derived iPSCs, where normal karyotypes of two iPSC lines (BC1 and PC1) derived by a single plasmid (pEB-C5) can be observed. The invention also provides PCR detection of episomal DNA. The pEB-Tg plasmid is used as a DNA control in a diluted amount equivalent to 1, 0.2 or 0.04 copies per genome of cellular DNA. The invention also provides in vitro pluripotency test via embryoid body (EB) formation and differentiation. Differentiated cell types derived from ectoderm (beta-3-tubulin), mesoderm (smooth muscle actin) and endoderm (AFP) can be found. Both BC1 and PC1 iPSCs can show undifferentiated and pluripotent phenotypes, have normal karyotypes, are essentially free of plasmid DNA by passage 12, and are pluripotent by a differentiation assay.

The invention provides characterization of iPSC line CTN4 derived from CB CD34+ cells reprogrammed by the pEB-C5 and pEB-Tg plasmids in the presence of sodium butyrate (NaB). The invention provides staining of undifferentiated CTN4 iPSCs after expansion (>5 passages) for marker associated with pluripotency. The invention also provides in vitro pluripotency assay by embryoid body (EB) formation and differentiation. Eight days after culture in suspension, EBs can be allowed to adhere and further differentiate. Then the whole culture was stained by monoclonal antibodies recognizing various cells types including those from ectoderm (β3-tubulin), mesoderm (smooth muscle actin) and endoderm (alpha-fetal protein or AFP). DAPI was used to stain cellular DNA. The invention provides in vivo pluripotency assay by teratoma formation. The cystic teratoma-like tumors were excised from animals and sectioned. H & E stained sections were examined. Both low (4×) and high (20×) magnification images of multiple sections can be observed. Various cell types such as neural rosettes (ectoderm), adipose (mesoderm) and glandular structures (endoderm) can be found.

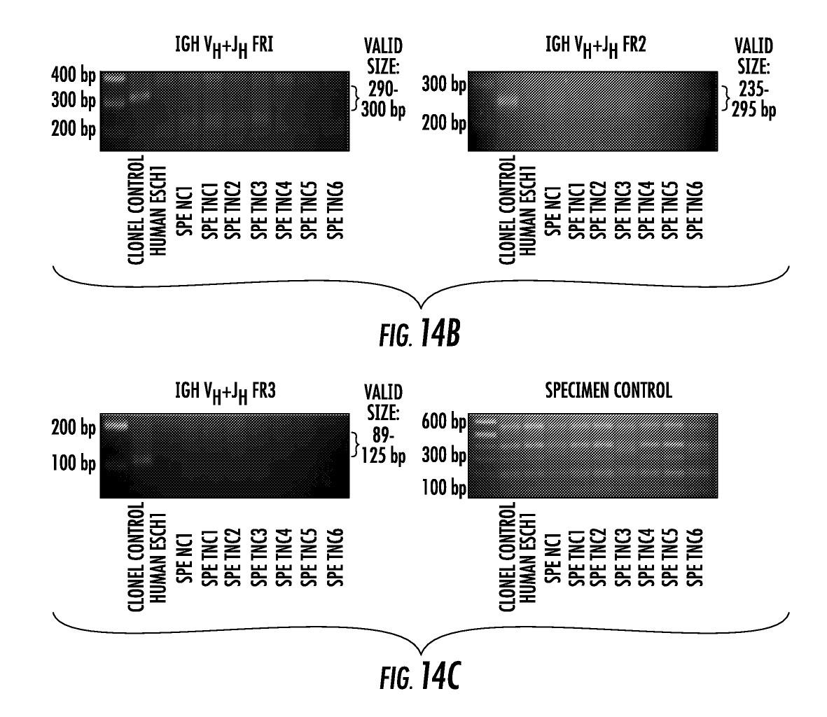

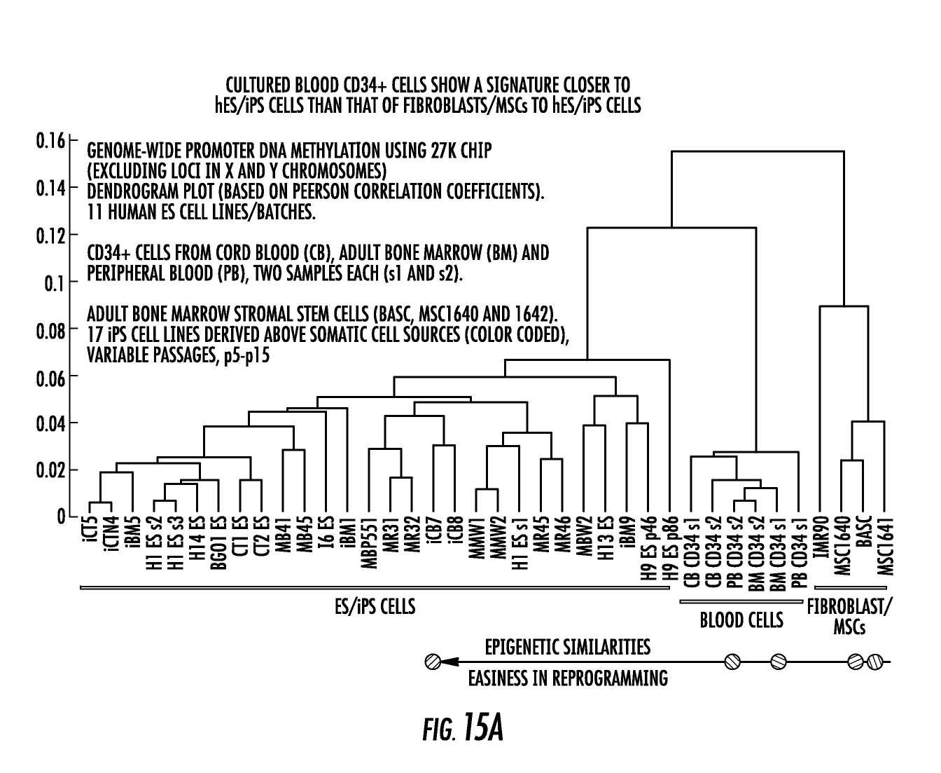

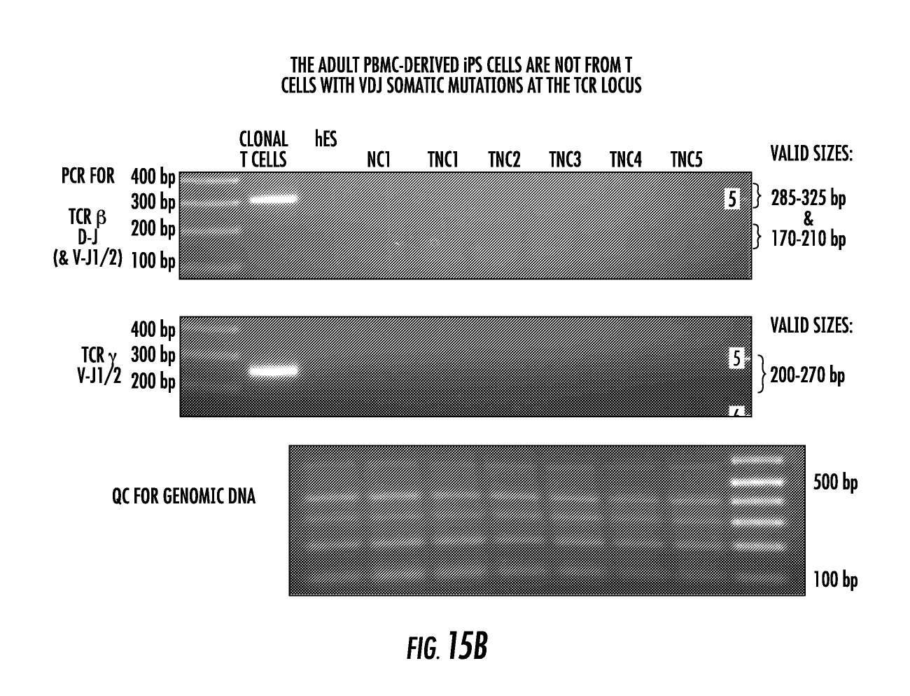

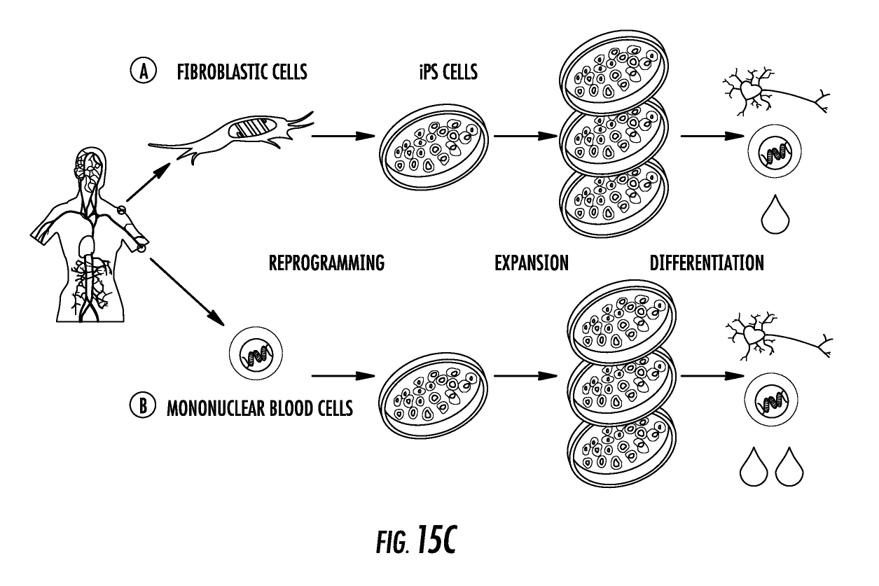

The inventor further characterized iPSCs derived from MNCs from adult PB MNCs especially from SCD003 (called SPE for Sickle PB Erythroblast), as with CD34+ cells shown previously. Using 4 sets of PCR primers for detecting rearranged TCR locus, and 3 sets of primers for rearranged IgH in B cells (FIG. 14), no evidence of somatic arrangement in iPSC clone NC#1 (by pEB-C5 vector alone) or 6 other clones (TNC#1-6, by pEB-Tg+pEB-C5 vectors) is observed. In the standard arrays, these clones after expansion display typical iPSC morphology and marker expression, are karyotypically normal and able to differentiate into the 3 embryonic germlayers. The invention provides undifferentiated phenotypes of an iPSC clone (SPE NC1) derived from SCD003 PB MNCs by the pEB-C5 (C5) vector alone+NaB. Other expanded and characterized clones such as TNC1 derived by using two plasmids (C5+Tg) are also provided. Normal karyotypes of 3 iPSC lines can be observed (NC1, TCN1 and TNC2) derived by a single plasmid (C5) or two plasmids (C5+Tg). When being analyzed for the status of episomal vector after reprogramming and expansion in these validated iPSC clones, the invention provides that the episomal DNA is gradually diluted and undetectable after 11-12 passages. In summary, the invention provides an efficient method for generating human iPSCs with an unaltered genome by using 1-2 episomal vectors to transfect primed adult PB or CB MNCs after 8-9 days of culture.

The invention further provides characterization of iPSC lines C7 and CN1 derived from CB CD34+ cells by the single pEB-C5 plasmid. The invention provides staining of undifferentiated C7 iPSCs after expansion (>5 passages) for marker associated with pluripotency. AP: alkaline phosphatase (histo-chemical staining). The invention also provides immuno-fluorescence staining with specific antibodies followed by Alexa555-conjugated secondary antibodies. Normal karyotypes of the iPSC line C7 and an additional line CN1 which was reprogrammed in the presence of sodium butyrate (NaB) can be observed. The invention further provides Giemsa staining of the cells cultured for 1 day or 9 days, after spun on slides, where morphology of expanded mononuclear cells (MNCs) from adult peripheral blood (PB) can be observed.

The invention provides reprogrammed colonies from un-fractionated adult peripheral blood mononuclear cells (PB MNCs) 9 days after expansion. MNCs isolated from phlebotomized blood of an adult sickle cell patient were cultured for 9 days. When 2×10⁶ expanded cells were nucleofected with 1-2 EBNA1/OriP plasmids, the inventor observed TRA-1-60+ colonies resembling iPSC candidate clones after day 14. Live TRA-1-60+ staining at day 20 (before the colonies were picked) can be observed.

### Example 13

### Teratoma Formation Assay of Pluripotency

The use of immuno-deficient mice for the teratoma formation assay was approved by the Animal Care and Use Committee at Johns Hopkins University. Three to five million iPSCs were harvested by Collagenase IV (Sigma) digestion (from one plates of 6-well plates), washed with PBS and resuspended in 200 μL diluted (1:1) ESC qualified Matrigel (354277, BD) solution. Cells were injected intra-muscularly into Rag1^{-/-}γC^{-/-} mice or NOD/SCID/IL2RG(γ_{c})^{-/-} mice or other improved immuno-deficient mice with a further reduced level of natural killer cells. Teratomas were excised 5-10 weeks after injection. After sectioning, slides containing various regions of teratomas were stained by H&E. Complex structures with various cell types were examined at both low and high magnitude.

### Example 14

### Epigenetic Analysis and Genome-Wide SNP Assay to Assess Genome Integrity

Epigenetic Analysis: The DNA methylation data was generated using the Infinium Human DNA Methylation27 BeadArray platform (Illumina, San Diego, Calif.). This platform allows us to interrogate DNA methylation of 27,578 informative CpG sites at single-nucleotide resolution, with 1 μg of genomic DNA. The selected CpG sites are located close to the promoter region of 14,475 RefSeq genes. The fully methylated CpG at these loci near the promoter often correlates with inactive expression of nearly genes (Mali et al. (2010) Stem Cells 28:713-20). Using this platform, the invention provides analysis of human CD34+ cells from CB, adult BM and PB (2 samples each), fetal fibroblastic (IMR90) and adult bone marrow derived stromal cells (3 samples), 17 iPSC lines derived from these somatic cells by various vectors (Ye et al. (2009) Blood 114:5473-80; Mali et al. (2010) Stem Cells 28:713-20), and 11 human ESC samples (8 independent lines and multiple batches or passages of H1 and H9). All analysis and data visualization was performed using MATLAB (The Math-Works, Natick, Mass.). Pearson-correlation coefficients, K-means clustering and classical multidimensional scaling (CMDS) was used to categorize the genes into various clusters and analyze their ensemble dynamics. For the Pearson-correlation based analyses all 27,578 loci were used, while for K-means clustering, 1,091 loci located in X and Y chromosomes were excluded to avoid scoring sex-specific differences. CMDS was performed on a list of Polycomb target genes, which are obtained from Ben-Porath et al. (2008) Nature Genetics 40:499-507).

Genome-wide SNP assay to assess genome integrity: The Illumina's Omni1_Quad BeadArray chip (Illumina) containing >10⁶ informative SNPs was used. The array analysis was performed by the Johns Hopkins SNP Center as part of Center for Inherited Disease Research (CIDR at the world wide web cidr.org). Based on 1,140,419 SNPs identified and the results with control (CEPH) genomic DNA samples that have been previously sequenced and included in each run, the Johns Hopkins SNP Center reported a 0.27% genotyping error rate in this run, within the normal range. Genomic DNA (˜2 μg) from CT5 and CTN4 iPSCs (p7) that are derived from CB CD34+ cells, and CD34− cells of the original CB donor were analyzed.

Although the invention has been described with reference to the above examples, it will be understood that modifications and variations are encompassed within the spirit and scope of the invention. Accordingly, the invention is limited only by the following claims.

What is claimed is:

1. A method for generating a pluripotent or multipotent stem cell comprising:
introducing a non-viral vector containing at least one pluripotency factor into a blood cell in culture, thereby reprogramming the blood cell to a pluripotent or multipotent stem cell.

2. The method of claim 1, wherein the vector comprises a plurality of pluripotency genes.

3. (canceled)

4. (canceled)

5. (canceled)

6. The method of claim 1, further comprising adding at least one cytokine to the culture.

7. The method of claim 6, wherein the at least one cytokine is selected from the group consisting of sertoli cell factor (SCF), Fms-related tyrosine kinase 3 ligand (FLT3 or FL), thrombopoietin (TPO), erythropoietin (EPO), and interleukin 3 (IL-3).

8. The method of claim 1, wherein the blood cells are cord blood (CB) cells, adult bone marrow (BM) CD34+ cells, adult peripheral blood (PB) cells, adult peripheral blood (PB) CD34+ cells, adult peripheral blood (PB) CD34+CD45+ cells, or adult peripheral blood mononuclear cells (PBMCs).

9. The method of claim 1, wherein the blood cell is from a mammalian subject.

10. The method of claim 9, wherein the subject is human.

11. The method of claim 1, wherein the pluripotent or multipotent stem cells comprise hematopoietic stem cells.

12. The method of claim 1, wherein the blood cell is from a subject having myeloproliferative disorders (MPDs).

13. (canceled)

14. (canceled)

15. The method of claim 1, wherein the at least one factor comprises OCT4, SOX2, KLF4, MYC, LIN28, and SV40 T antigen, or active fragments thereof

16. The method of claim 1 wherein the non-viral vector is an episomal vector.

17. The method of claim 16, further comprising introducing a second episomal vector into the blood cells.

18. The method of claim 17, wherein the second episomal vector expresses SV40 T antigen (Tg).

19. The method of claim 16, wherein the first episomal vector comprises a plurality of pluripotency genes operatively linked to at least one regulatory sequence for expressing the factors.

20. The method of claim 16, wherein the episomal vector is a oriP/EBNA1 plasmid.

21. The method of claim 8, wherein the PBMCs are from a sickle cell anemia patient.

22. (canceled)

23. A method of identifying an agent having a therapeutic effect on cells of a subject, comprising contacting the pluripotent or multipotent stem cells of claim 1 with a test agent, and detecting a change in a function in presence of the test agent as compared to the function in absence of the test agent.

24. A method of generating differentiated cells, comprising inducing differentiation of the pluripotent or multipotent stem cells produced by the method of claim 1, thereby obtaining a population of differentiated cells.

25. The method of claim 24, wherein the differentiated cells comprise blood cells, muscle cells, neuronal cells, connective tissues, cardiomyocyte cells, megakaryocyte cells, endothelial cells, hepatocytes, nephrogenic cells, adipogenic cells, osteoblast cells, osteoclastic cells, alveolar cells, cardiac cells, intestinal cells, renal cells, retinal cells or epithelial cells.

26. The method of claim 24, wherein the differentiated cells comprise pancreatic beta cells, hepatocytes, cardiomyocytes, or skeletal muscle cells.

27. An enriched population of isolated pluripotent or multipotent stem cells produced by the method of claim 1.

28. (canceled)

29. A method of treating a disease requiring replacement or renewal of cells comprising administering to a subject an effective amount of the pluripotent or multipotent stem cells generated according to claim 1.

30. A reprogramming non-viral episomal vector comprising at least five pluripotency genes operatively linked to at least one regulatory sequence for expressing at least five pluripotency factors.

31. The vector of claim 30, wherein the vector is a oriP/EBNA1 plasmid.


SEQUENCE LISTING (4 sequences)

SEQ ID NO: 1 - DNA, 22 bp, Artificial Sequence
  Primer
tttaatacgattgagggcgtct

SEQ ID NO: 2 - DNA, 22 bp, Artificial Sequence
  Primer
ggttttgaaggatgcgattaag

SEQ ID NO: 3 - DNA, 20 bp, Artificial Sequence
  Primer
gccaggtgggttaaaggagc

SEQ ID NO: 4 - DNA, 23 bp, Artificial Sequence
  Primer
ggtacttatagtggctgggctgt